(19) United States
(12) Patent Application Publication
(10) Pub. No.: US 2010/0028904 A1
(43) Pub. Date: Feb. 4, 2010

(54) WAY TO OBTAIN HIGH EXPRESSION CLONES OF MAMMALIAN CELLS USING A METHYLCELLULOSE WITH FLUORESCENT PROTEIN A OR G AND FLUORESCENT SCREENING METHOD

(75) Inventors: Edward Appelbaum, Radnor, PA (US); Susanne Corisdeo, Radnor, PA (US); Subiney Ganguly, Newtown, PA (US); Dennis M. Kraichely, Radnor, PA (US); Sunil Mehta, Morrisville, NC (US); Gordon Moore, Radnor, PA (US); Richard Siegel, Horsham, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: CENTOCOR, INC., Malvern, PA (US)

(21) Appl. No.: 12/530,546

(22) PCT Filed: Mar. 28, 2008
(86) PCT No.: PCT/US2008/058561

§ 371 Date: Sep. 9, 2009

Related U.S. Application Data
(60) Provisional application No. 60/909,097, filed on Mar. 30, 2007.

(51) Int. Cl. G01N 33/53; C12Q 1/02; C12N 5/12; C12N 1/19; C12N 5/073; C12N 1/21; C12N 1/12

(52) U.S. Cl. 435/7.1; 435/29; 435/325; 435/348; 435/254.2; 435/365; 435/369; 435/358; 435/367; 435/252.3; 435/257.2

(57) ABSTRACT

The invention provides a genetic screening method for identifying a transfected cell expressing the polypeptide of interest. The methods allows for high throughput screening of recombinant cells for elevated levels of expression of the polypeptide of interest using methylcellulose comprising fluorescent protein A or G to improve detection and cloning. The invention also provides capture media, formulations and methods of making and using thereof.

This application claims the benefit of International Application Number PCT/US PCT/US2008/058561 filed Mar. 28, 2008, which claims priority to U.S. Prov. Appl. No. 60/909,097, filed Mar. 30, 2007 and which is entirely incorporated herein by reference.

### BACKGROUND OF THE INVENTION

1. Field of the Invention

The present invention pertains to genetic screening methods, related cells and culturing media thereof, useful in identifying clones of mammalian cells expressing the polypeptide of interest. The methods allows for high throughput screening of recombinant cells for elevated levels of expression of polypeptide of interest. The present invention also provides a screening method useful in screening and isolating clones of mammalian cells expressing high levels of immunoglobulin.

2. Related Background

Recombinant proteins (r-proteins) are an emerging class of therapeutic agents. To obtain a stable clone for recombinant protein production usually requires the transfection of cells with an expression vector containing gene of interest and a dominant genetic marker.

Typically, for the selection of stable transfectants, a selectable marker such as an antibiotic resistance gene is transfected along with the target gene of interest. Selection is then carried out in the presence of the specific antibiotic. Cells that have taken up the expression vector DNA survive in appropriate selection media.

Currently, cloning of stably transfected cells relies on performing a series of limiting dilution procedures, a time consuming and labor-intensive process. For example, many commonly used mammalian expression systems are based on stably transfected Chinese Hamster Ovary (CHO) cells and transfection efficiencies in this system range from 10-60% of cells taking up the vector DNA. However, a wide variation in recombinant gene expression exists among clones that stably incorporate the foreign DNA into the genome due to the position effect by which different regions of the chromosome modulate the expression of the transfected gene. Many hundreds, even thousands of transfected clones are typically screened for random high producers because of the random variation in recombinant protein production. Therefore in many cases, screening for high producers has been one of the rate limiting procedures in developing of cell lines expressing r-proteins due to the huge amount of cells to screen and the complicated assays to perform.

Soluble proteins interact with their corresponding antibody to form a precipitate in solid or semisolid substrates such as agarose. One such application is the immunoplate assay used to detect mouse myeloma mutants. Briefly, cells are cloned in soft agarose over feeder layers that undergo contact inhibition. Antibody or antigen reactive with the immunoglobulin that is secreted by the cloned cells is added to the plate and diffuses through the agarose forming an antigen-antibody precipitate surrounding the clone. This precipitate appears as a collection of dark granules and specks under low or medium power with an inverted microscope. This assay was used not only to look for mutants of hybridoma and myeloma cells, but also to clone hybridomas and identify subclones producing the desired antibody. It can also be used to identify high producers.

However, several difficulties were reported previously when using this semi-solid agarose technique for screening clones producing the desired antibody. For example, poor growth of mammalian cells is caused by inability to utilize the correct temperature to seed cells while agarose is cooling. Another common problem is the difficulty in viewing the precipitate in the agarose media even under a microscope. It is also difficult to correlate the precipitate size to the level of protein secretion.

Recombinant protein production entails generation of a clonal cell line that expresses large amounts of recombinant protein. Generation of high-producer clones requires an assay that can quantitatively measure protein relative to other clones and that can effectively isolate it from low-producers. It is a recognized challenge to have both of these important features combined in a single assay. Although Fluorescent activated cell sorter (FACS) and Halo (United States Patent Application 20050118652A1) procedures combine both features, FACS is associated with decreased survival rate of isolated clones and Halo method uses rabbit anti-sera, which requires additional testing for rabbit viruses on selected cell lines. Furthermore, the Halo procedure is only partially predictive and may require screening of a larger number of clones. In other widely used procedures, clones are first separated and then an assay is used to quantify recombinant protein.

Accordingly, there is a need to provide improved and/or modified screening methods, which overcome and/or substantially ameliorate one or more of these and other problems known in the art.

### BRIEF DESCRIPTION OF THE DRAWINGS

FIG. 1A-B. FIG. 1A is a photograph of representative halo-producing cell. 1B is a photograph of a representative improvement showing of a fluorescent protein A or G halo-producing cell. FIG. 1: Example of fluorescent protein G based secreted protein detection assay. Photographs were taken on day 11. Final concentration of Alexa Fluor 488 protein G is 16 ug/mL. Picture on left shows fluorescent colonies while image on right shows all colonies. Non-fluorescent colonies are circled.

FIG. 2A-B. FIG. 2A is a photograph of representative halo-producing cell. 2B is a photograph of a representative improvement showing of a fluorescent protein A or G halo-producing cell. Example of fluorescent protein A based secreted protein detection assay. Photographs were taken on day 11. Final concentration of Alexa Fluor 488 protein A is 13 ug/mL. Picture on left shows fluorescent colonies while image on right shows all colonies. Non-fluorescent colonies are circled.

FIG. 3 is a graphical representation showing the correlation between batch shake flask overgrowth titer and total fluorescence.

FIG. 4A is a graphical representation of 48 colonies from each condition with the highest fluorescence intensity that were selected and expanded to 24-well cultures for overgrowth titer determination

FIG. 4B is a graphical representation of 24-well overgrowth titers for top six sub-clone cell lines in Example 2 were determined to range from 450-600 mg/L

FIG. 5A is a graphical representation of clones that were expanded to 24-well cultures. 24-well overgrowth titers ranged from 0-18 mg/L

FIG. 5B is a graphical representation of 24-well titers overgrowth, where the top 10 highest expressing clones were selected for expansion to shake flasks. Shake flask overgrowth titers ranged from 0-120 mg/L (MACH-1).

FIG. 6A is a graphical representation of 24-well overgrowth titers of 48 clones expanded to 24-well cultures ranged from 0-65 mg/L, including an outlier clone producing 65 mg/L

FIG. 6B is a graphical representation of batch shake flask overgrowth titers were determined for the top 10 cell lines ranged from 0-330 mg/L (MACH-1).

### SUMMARY OF THE INVENTION

The present invention relates to improved genetic screening methods, related cells and culturing media thereof, useful in identifying and/or characterizing clones of mammalian cells expressing the polypeptide of interest. The methods allow for high throughput screening of recombinant cells for elevated levels of expression of polypeptide of interest using methylcellulose comprising fluorescent protein A or G.

A procedure to identify high-producing clones is invented. Cells expressing recombinant protein (with affinity for protein A and/or protein G) plated in a semi-solid media containing fluorescent Protein A or Protein G produce fluorescence on the surface and around the cell colonies. Total fluorescence on a cell colony and its surrounding is directly proportional to the amount of secreted protein. This procedure has the ability to effectively differentiate clones that are high-producers from low-producers or parental cells. Therefore, this method reduces the screening effort without compromising the outcome.

In one embodiment, the present invention provides a method for selecting high expression cell clones expressing a polypeptide of interest, comprising: (a) selecting high expression cell clones among cells cultured in a semi-solid culture medium comprising fluorescent protein A or G and expressing said polypeptide of interest, wherein the level of fluorescence from the fluorescent Protein A or G indicates the relative expression of said polypeptide for each cell or group of cells. In addition, the present invention further relates to a cell clone identified by such a method.

The cells may be any cell type including prokaryotic and eukaryotic cells. Prokaryotic cells may include but are not limited to bacterial cells or blue-green algae cells. Eukaryotic cells may include but are not limited to mammalian cells, yeast cells or insect cells. Preferably, the cells are eukaryotic cells. In a preferred embodiment, suitable cell lines that can be used according to the present invention include any transformed or immortalized mammalian cell line. Such cell lines include myeloma cell lines, such as Sp2/0, NSO, NS1, CHO, BHK, Ag653, P3X63Ag8.653 cells (ATCC Accession Number CRL-1580) and SP2/0-Ag14 cells (ATCC Accession Number CRL-1851), COS-1 (e.g., ATCC CRL 1650), COS-7 (e.g., ATCC CRL-1651), HEK293, BHK21 (e.g., ATCC CAL-10), CHO (e.g., ATCC CRL 1610, CHO DXB-11, CHO DG44), BSC-1 (e.g., ATCC CAL-26) cell lines, HepG2 cells, P3X63Ag8.653, 293 cells, HeLa cells, NIH 3T3, CDS-1, CDS-7, NIH 273, and the like, or any cells derived therefrom, including cell fusions of the above, such as to protein producing cells, such as B-cells, antibody producing cells, isolated or cloned spleen or lymph node cells, and the like.

The present invention further provides a method of isolating a polypeptide of interest comprising, in addition to above mentioned step (a), harvesting and culturing the cell clones; and isolating the polypeptide of interest therefrom. Moreover, the present invention further relates to at least one polypeptide of interest isolated by such a method.

The polypeptide of interest may be any suitable soluble or membrane-bound polypeptide including, for example but not limited to, an antibody, a growth factor, a hormone, a biopharmaceutical, a receptor or a synthetic polypeptide of interest or portions thereof.

In a preferred embodiment, the polypeptide of interest is a diagnostic or a therapeutic protein. The diagnostic or therapeutic protein may be an immunoglobulin, a cytokine, an integrin, an antigen, a growth factor, a receptor or fusion protein thereof, any fragment thereof, or any structural or functional analog thereof. The diagnostic or therapeutic protein may also be a cell cycle protein, a hormone, a neurotransmitter, a blood protein, an antimicrobial, any fragment thereof, or any structural or functional analog thereof.

In a preferred embodiment, the cell clones selected using the method of the present invention may produce an immunoglobulin or fragment thereof derived from a rodent or a primate. Alternatively, the immunoglobulin or fragment thereof may be chimeric or engineered. Indeed, the present invention further contemplates methods of identifying cell clones that express an immunoglobulin or fragment thereof which is humanized, CDR grafted, phage displayed, transgenic mouse-produced, optimized, mutagenized, randomized or recombined.

The immunoglobulin or fragment thereof may include, but not limited to, IgG1, IgG2, IgG3, IgG4, IgA1, IgA2, IgD, IgE, IgM, and any structural or functional analog thereof. In a specific embodiment, the immunoglobulin expressed in the cells, cell lines, and cell cultures of the present invention is infliximab, a chimeric anti-TNF alpha antibody. Furthermore, the immunoglobulin fragment isolated using the method of the present invention may include, but is not limited to, F(ab′)₂, Fab′, Fab, Fc, Facb, Fc′, Fd, Fv and any structural or functional analog thereof. In a specific embodiment, the immunoglobulin fragment is abciximab.

The polypeptide of interest may further include, but not limited to an antigen, a cytokine, an integrin, an antigen, a growth factor, a hormone, a neurotransmitter, a receptor or fusion protein thereof, a blood protein, an antimicrobial, any fragment thereof, and any structural or functional analog of any of the foregoing.

In one embodiment of the present invention, the polypeptide of interest is an integrin. Examples of integrins contemplated by the present invention include, but are not limited to, α1, α2, α3, α4, α5, α6, α7, α8, α9, αD, αL, αM, αV, αX, αIIb, αIELb, β1, β2, β3, β4, β5, β6, β7, β8, α1β1, α2β1, α3β1, α4β1, α5β1, α6β1, α7β1, α8β1, α9β1, α4β7, α6β4, αDβ2, αLβ2, αMβ2, αVβ1, αVβ3, αVβ5, αVβ6, αVβ8, αXβ2, αIIbβ3, αIELbβ7, and any structural or functional analog thereof.

In an embodiment of the present invention, the polypeptide of interest is an antigen.

The antigen may be derived from a number of sources including, but not limited to, a bacterium, a virus, a blood protein, a cancer cell marker, a prion, a fungus, and any structural or functional analog thereof.

In yet another embodiment, the polypeptide of interest is a growth factor. Examples of the growth factors contemplated by the present invention include, but are not limited to, a human growth factor, a platelet derived growth factor, an epidermal growth factor, a fibroblast growth factor, a nerve growth factor, a chorionic gonadotropin, an erythrpoeitin, an activin, an inhibin, a bone morphogenic protein, a transforming growth factor, an insulin-like growth factor, and any structural or functional analog thereof.

In yet another embodiment, the polypeptide of interest is a cytokine. Examples of cytokines contemplated by the present invention include, but are not limited to, an interleukin, an interferon, a colony stimulating factor, a tumor necrosis factor, an adhesion molecule, an angiogenin, an annexin, a chemokine, and any structural or functional analog thereof.

In another embodiment, the polypeptide of interest is a growth hormone. The growth hormone may include, but is not limited to, a human growth hormone, a prolactin, a follicle stimulating hormone, a chorionic gonadotrophin, a leuteinizing hormone, a thyroid stimulating hormone, a parathyroid hormone, an estrogen, a progesterone, a testosterone, an insulin, a proinsulin, and any structural or functional analog thereof.

The present invention further relates to the expression of neurotransmitters using the method taught herein. Examples of neurotransmitters include, but are not limited to, an endorphin, a coricotropin releasing hormone, an adrenocorticotropic hormone, a vaseopressin, a giractide, an N-acytlaspartylglutamate, a peptide neurotransmitter derived from pre-opiomelanocortin, any antagonists thereof, and any agonists thereof.

In another embodiment, the polypeptide of interest is a receptor or fusion protein. The receptor or fusion protein may be, but is not limited to, an interleukin-1, an interleukin-12, a tumor necrosis factor, an erythropoietin, a tissue plasminogen activator, a thrombopoetin, and any structural or functional analog thereof.

Alternatively, recombinant blood proteins may be isolated by the method of the present invention. Such recombinant proteins include, but are not limited to, an erythropoietin, a thrombopoeitin, a tissue plasminogen activator, a fibrinogen, a hemoglobin, a transferrin, an albumin, a protein c, and any structural or functional analog thereof.

In another embodiment, the polypeptide of interest is a recombinant antimicrobial agent. Examples of antimicrobial agents contemplated by the present invention include, for example, a beta-lactam, an aminoglycoside, a polypeptide antibiotic, and any structural or functional analog thereof.

The present invention further provides semi-solid capture medium comprising cell growth medium, a gelatinization agent comprising fluorescent protein A or G. The gelatinization agent may be any polymer that when dissolved in an aqueous cell growth medium, forms semi-solid gel under the temperature suitable for culturing cells. The gelatinization agent may be selected from, but not limited to, agar, agarose, methylcellulose, matrigel, collagen, gelatin, or other similar materials. Preferably, the gelatinization agent is methylcellulose. Such media composition and formulation of the present invention allow the identification of cells expressing the polypeptide of interest by monitoring the precipitate halo formed between the polypeptide of interest and the capture molecule which detection is enhanced by using gelatinization agents comprising fluorescent protein A or G. Accordingly the present invention provides specific media, formulations and methods of making and using thereof.

### DESCRIPTION OF THE INVENTION

For many commonly used mammalian expression systems, cloning of stably transfected cells is a time consuming and labor-intensive process. Many hundreds, even thousands of transfected clones are typically screened for high producers because of the random variation in recombinant protein production. The present invention relates to an improved, rapid way to screen for clones producing high levels of polypeptide of interest. The method is based on the flourescence formed between the polypeptide of interest and bound fluorescent Protein A or Protein G, receptor and/or ligand in a semi-solid detection or capture medium comprising fluorescent protein A or G that floresces when bound to the polypeptide of interest.

For example, when cells expressing a recombinant protein are plated in methylcellulose media containing fluorescent protein A or protein G, fluorescence is visible on the cell colonies and around them. The concentration of fluorescent protein A or protein G on or around a cell colony is directly proportional to the amount of protein secreted from the cell colony. Complex formation between protein A or protein G and secreted protein leads to reduction in free protein A or Protein G around recombinant-protein-producing cell colonies. Equilibrium is established by spontaneous diffusion of free protein A or protein G to region surrounding the cell colonies. Overall, this results in high amounts of fluorescent protein A or protein G on or around the recombinant-protein-producing cell colony.

It is well known in the art that if the transfected cells have been in continuous culture for a long time, or the cells in culture are not derived from a single cell clone, they may need to be recloned. The present invention also provides a method to rapidly achieve this goal.

In one embodiment of the present invention, methods are provided for selecting high expression cell clones expressing a polypeptide of interest, comprising: (a) selecting high expression cell clones among cells cultured in a semi-solid culture medium comprising fluorescent protein A or G and expressing said polypeptide of interest, wherein said cells are contacted with fluorescent protein A or G that interacts with the polypeptide of interest such that said level of flourescence indicates relative expression of said polypeptide for each cell or group of cells. In a preferred embodiment, the semi-solid capture medium is methylcellulose or agar based.

In another embodiment, the present invention provides a method of isolating a polypeptide of interest comprising the steps in addition to above mentioned (a), harvesting and culturing the cell clone; and isolating the polypeptide of interest therefrom.

### Polypeptides of Interest

The polypeptides of interest include, but are not limited to, immunoglobulins, integrins, antigens, growth factors, cell cycle proteins, cytokines, hormones, neurotransmitters, receptor or fusion proteins thereof, blood proteins, antimicrobials, or fragments, or structural or functional analogs thereof. These following descriptions do not serve to limit the scope of the invention, but rather illustrate the breadth of the invention.

For example, in one embodiment of the invention, the immunoglobulin may be derived from human or non-human polyclonal or monoclonal antibodies. Specifically, these immunoglobulins (antibodies) may be recombinant and/or synthetic human, primate, rodent, mammalian, chimeric, humanized or CDR-grafted, antibodies and anti-idiotype antibodies thereto. These antibodies can also be produced in a variety of truncated forms in which various portions of antibodies are joined together using genetic engineering techniques. As used presently, an “antibody,” “antibody fragment,” “antibody variant,” “Fab,” and the like, include any protein- or peptide-containing molecule that comprises at least a portion of an immunoglobulin molecule, such as but not limited to at least one CDR of a heavy or light chain or a ligand binding portion thereof, a heavy chain or light chain variable region, a heavy chain or light chain constant region, a framework region, or any portion thereof, which may be expressed in the cell culture of the present invention. Such antibodies optionally further affect a specific ligand, such as but not limited to, where such antibody modulates, decreases, increases, antagonizes, agonizes, mitigates, alleviates, blocks, inhibits, abrogates and/or interferes with at least one target activity or binding, or with receptor activity or binding, in vitro, in situ and/or in vivo.

In one embodiment of the invention, such antibodies, or functional equivalents thereof, may be “human,” such that they are substantially non-immunogenic in humans. These antibodies may be prepared through any of the methodologies described herein or well know in the art.

The term “antibody” is further intended to encompass antibodies, digestion fragments, specified portions and variants thereof, including antibody mimetics or comprising portions of antibodies that mimic the structure and/or function of an antibody or specified fragment or portion thereof, including single chain antibodies and fragments thereof, that are expressed in the cell culture of the present invention. The present invention thus encompasses antibody fragments capable of binding to a biological molecule (such as an antigen or receptor) or portions thereof, including but not limited to Fab (e.g., by papain digestion), Fab′ (e.g., by pepsin digestion and partial reduction) and F(ab′)₂ (e.g., by pepsin digestion), facb (e.g., by plasmin digestion), pFc′ (e.g., by pepsin or plasmin digestion), Fd (e.g., by pepsin digestion, partial reduction and reaggregation), Fv or scFv (e.g., by molecular biology techniques) fragments. See, e.g., Current Protocols in Immunology, (Coligan et al., John Wiley & Sons, Inc., NY, N.Y. 1992-2007).

The nature and source of the polypeptide of interest expressed in the cell clones of the present invention are not limited. The following is a general discussion of the variety of proteins, peptides and biological molecules that may be used in the in accordance with the teachings herein. These descriptions do not serve to limit the scope of the invention, but rather illustrate the breadth of the invention.

Thus, an embodiment of the present invention may include the production of one or more growth factors. Briefly, growth factors are hormones or cytokine proteins that bind to receptors on the cell surface, with the primary result of activating cellular proliferation and/or differentiation. Many growth factors are quite versatile, stimulating cellular division in numerous different cell types; while others are specific to a particular cell-type. The following Table 1 presents several factors, but is not intended to be comprehensive or complete, yet introduces some of the more commonly known factors and their principal activities.

TABLE 1
Growth Factors

| Factor | Principal Source | Primary Activity | Comments |
|---|---|---|---|
| Platelet Derived | Platelets, endothelial | Promotes proliferation of | Dimer required for |
| Growth Factor | cells, placenta. | connective tissue, glial and | receptor binding. |
| (PDGF) | | smooth muscle cells. PDGF | Two different |
| | | receptor has intrinsic | protein chains, A |
| | | tyrosine kinase activity. | and B, form 3 |
| | | | distinct dimer |
| | | | forms. |
| Epidermal | Submaxillary gland, | promotes proliferation of | EGF receptor has |
| Growth Factor | Brunners gland. | mesenchymal, glial and | tyrosine kinase |
| (EGF) | | epithelial cells. | activity, activated |
| | | | in response to EGF |
| | | | binding. |
| Fibroblast | Wide range of cells; | Promotes proliferation of | Four distinct |
| Growth Factor | protein is associated | many cells including | receptors, all with |
| (FGF) | with the ECM; nineteen | skeletal and nervous system; | tyrosine kinase |
| | family members. | inhibits some stem cells; | activity. FGF |
| | Receptors widely | induces mesodermal | implicated in |
| | distributed in bone, | differentiation. Non- | mouse mammary |
| | implicated in several | proliferative effects include | tumors and |
| | bone-related diseases. | regulation of pituitary and | Kaposi's sarcoma. |
| | | ovarian cell function. | |
| NGF | | Promotes neurite outgrowth | Several related |
| | | and neural cell survival. | proteins first |
| | | | identified as proto- |
| | | | oncogenes; trkA |
| | | | (trackA), trkB, |
| | | | trkC. |
| Erythropoietin | Kidney. | Promotes proliferation and | Also considered a |
| (Epo) | | differentiation of | ‘blood protein,’ and |
| | | erythrocytes. | a colony |
| | | | stimulating factor. |
| Transforming | Common in | Potent keratinocyte growth | Related to EGF. |
| Growth Factor a | transformed cells, | factor. | |
| (TGF-a) | found in macrophages | | |
| | and keratinocytes. | | |
| Transforming | Tumor cells, activated | Anti-inflammatory | Large family of |
| Growth Factor v | TH₁ cells (T-helper) | (suppresses cytokine | proteins including |
| (TGF-b) | and natural killer (NK) | production and class II | activin, inhibin and |
| | cells. | MHC expression), | bone morpho- |
| | | proliferative effects on | genetic protein. |
| | | many mesenchymal and | Several classes and |
| | | epithelial cell types, may | subclasses of cell- |
| | | inhibit macrophage and | surface receptors. |
| | | lymphocyte proliferation. | |
| Insulin-Like | Primarily liver, | Promotes proliferation of | Related to IGF-II |
| Growth Factor-I | produced in response to | many cell types, autocrine | and proinsulin, also |
| (IGF-I) | GH and then induces | and paracrine activities in | called |
| | subsequent cellular | addition to the initially | Somatomedin C. |
| | activities, particularly | observed endocrine | IGF-I receptor, like |
| | on bone growth. | activities on bone. | the insulin receptor, |
| | | | has intrinsic |
| | | | tyrosine kinase |
| | | | activity. IGF-I can |
| | | | bind to the insulin |
| | | | receptor. |
| Insulin-Like | Expressed almost | Promotes proliferation of | IGF-II receptor is |
| Growth | exclusively in | many cell types primarily of | identical to the |
| Factor-II | embryonic and | fetal origin. Related to | mannose-6- |
| (IGF-II) | neonatal tissues. | IGF-I and proinsulin. | phosphate receptor |
| | | | that is responsible |
| | | | for the integration |
| | | | of lysosomal |
| | | | enzymes. |

Additional growth factors that may be produced in accordance with the present invention include Activin (Vale et al., 321 Nature 776 (1986); Ling et al., 321 Nature 779 (1986)), Inhibin (U.S. Pat. Nos. 4,737,578; 4,740,587), and Bone Morphongenic Proteins (BMPs) (U.S. Pat. No. 5,846,931; Wozney, Cellular & Molecular Biology of Bone 131-167 (1993)).

In addition to the growth factors discussed above, the present invention may target or use other cytokines. Secreted primarily from leukocytes, cytokines stimulate both the humoral and cellular immune responses, as well as the activation of phagocytic cells. Cytokines that are secreted from lymphocytes are termed lymphokines, whereas those secreted by monocytes or macrophages are termed monokines. A large family of cytokines are produced by various cells of the body. Many of the lymphokines are also known as interleukins (ILs), because they are not only secreted by leukocytes, growth factors targeted to cells of hematopoietic origin. The list of identified interleukins grows continuously. See, e.g. U.S. Pat. No. 6,174,995; U.S. Pat. No. 6,143,289; Sallusto et al., 18 Annu. Rev. Immunol. 593 (2000); Kunkel et al., 59 J. Leukocyto Biol. 81 (1996).

Additional growth factor/cytokines encompassed in the present invention include pituitary hormones such as human growth hormone (HGH), follicle stimulating hormones (FSH, FSHα, and FSHβ), Human Chorionic Gonadotrophins (HCG, HCGα, HCGβ), uFSH (urofollitropin), Gonatropin releasing hormone (GRH), Growth Hormone (GH), leuteinizing hormones (LH, LHα, LHβ), somatostatin, prolactin, thyrotropin (TSH, TSHα, TSHβ), thyrotropin releasing hormone (TRH), parathyroid hormones, estrogens, progesterones, testosterones, or structural or functional analog thereof. All of these proteins and peptides are known in the art.

The cytokine family also includes tumor necrosis factors, colony stimulating factors, and interferons. See, e.g., Cosman, 7 Blood Cell (1996); Gruss et al., 85 Blood 3378 (1995); Beutler et al., 7 Annu. Rev. Immunol. 625 (1989); Aggarwal et al., 260 J. Biol. Chem. 2345 (1985); Pennica et al., 312 Nature 724 (1984); R & D Systems, Cytokine Mini-Reviews, at http://www.rndsystems.com.

Several cytokines are introduced, briefly, in Table 2 below.

TABLE 2
Cytokines

| Cytokine | Principal Source | Primary Activity |
|---|---|---|
| Interleukins | Primarily macrophages but also | Costimulation of APCs and T cells; |
| IL1-a and -b | neutrophils, endothelial cells, | stimulates IL-2 receptor production |
| | smooth muscle cells, glial cells, | and expression of interferon-γ; may |
| | astrocytes, B- and T-cells, | induce proliferation in non-lymphoid |
| | fibroblasts, and keratinocytes | cells. |
| IL-2 | CD4+ T-helper cells, activated TH₁ | Major interleukin responsible for |
| | cells, NK cells | clonal T-cell proliferation. IL-2 also |
| | | exerts effects on B-cells, |
| | | macrophages, and natural killer (NK) |
| | | cells. IL-2 receptor is not expressed |
| | | on the surface of resting T-cells, but |
| | | expressed constitutively on NK cells, |
| | | that will secrete TNF-α, IFN-γ and |
| | | GM-CSF in response to IL-2, which in |
| | | turn activate macrophages. |
| IL-3 | Primarily T-cells | Also known as multi-CSF, as it |
| | | stimulates stem cells to produce all |
| | | forms of hematopoietic cells. |
| IL-4 | TH₂ and mast cells | B cell proliferation, eosinophil and |
| | | mast cell growth and function, IgE and |
| | | class II MHC expression on B cells, |
| | | inhibition of monokine production |
| IL-5 | TH₂ and mast cells | eosinophil growth and function |
| IL-6 | Macrophages, fibroblasts, | IL-6 acts in synergy with IL-1 and |
| | endothelial cells and activated T- | TNF-α in many immune responses, |
| | helper cells. Does not induce | including T-cell activation; primary |
| | cytokine expression. | inducer of the acute-phase response in |
| | | liver; enhances the differentiation of |
| | | B-cells and their consequent |
| | | production of immunoglobulin; |
| | | enhances Glucocorticoid synthesis. |
| IL-7 | thymic and marrow stromal cells | T and B lymphopoiesis |
| IL-8 | Monocytes, neutrophils, | Chemoattractant (chemokine) for |
| | macrophages, and NK cells | neutrophils, basophils and T-cells; |
| | | activates neutrophils to degranulate. |
| IL-9 | T cells | hematopoietic and thymopoietic |
| | | effects |
| IL-10 | activated TH₂ cells, CD8⁺ T and B | inhibits cytokine production, promotes |
| | cells, macrophages | B cell proliferation and antibody |
| | | production, suppresses cellular |
| | | immunity, mast cell growth |
| IL-11 | stromal cells | synergisitc hematopoietic and |
| | | thrombopoietic effects |
| IL-12 | B cells, macrophages | proliferation of NK cells, INF-g |
| | | production, promotes cell-mediated |
| | | immune functions |
| IL-13 | TH₂ cells | IL-4-like activities |
| IL-18 | macrophages/Kupffer cells, | Interferon-gamma-inducing factor |
| | keratinocytes, glucocorticoid- | with potent pro-inflammatory activity |
| | secreting adrenal cortex cells, and | |
| | osteoblasts | |
| IL-21 | Activated T cells | IL21 has a role in proliferation and |
| | | maturation of natural killer (NK) cell |
| | | populations from bone marrow, in the |
| | | proliferation of mature B-cell |
| | | populations co-stimulated with anti- |
| | | CD40, and in the proliferation of T |
| | | cells co-stimulated with anti-CD3. |
| IL-23 | Activated dendritic cells | A complex of p19 and the p40 subunit |
| | | of IL-12. IL-23 binds to IL-12R beta 1 |
| | | but not IL-12R beta 2; activates Stat4 |
| | | in PHA blast T cells; induces strong |
| | | proliferation of mouse memory T |
| | | cells; stimulates IFN-gamma |
| | | production and proliferation in PHA |
| | | blast T cells, as well as in CD45RO |
| | | (memory) T cells. |
| TumorNecrosis | Primarily activated macrophages. | Once called cachectin; induces the |
| Factor | | expression of other autocrine growth |
| TNF-α | | factors, increases cellular |
| | | responsiveness to growth factors; |
| | | induces signaling pathways that lead |
| | | to proliferation; induces expression of |
| | | a number of nuclear proto-oncogenes |
| | | as well as of several interleukins. |
| (TNF-β) | T-lymphocytes, particularly | Also called lymphotoxin; kills a |
| | cytotoxic T-lymphocytes (CTL | number of different cell types, induces |
| | cells); induced by IL-2 and antigen- | terminal differentiation in others; |
| | T-Cell receptor interactions. | inhibits lipoprotein lipase present on |
| | | the surface of vascular endothelial |
| | | cells. |
| Interferons | macrophages, neutrophils and some | Known as type I interferons; antiviral |
| INF-a and -b | somatic cells | effect; induction of class I MHC on all |
| | | somatic cells; activation of NK cells |
| | | and macrophages. |
| Interferon | Primarily CD8+ T-cells, activated | Type II interferon; induces of class I |
| INF-γ | TH₁ and NK cells | MHC on all somatic cells, induces |
| | | class II MHC on APCs and somatic |
| | | cells, activates macrophages, |
| | | neutrophils, NK cells, promotes cell- |
| | | mediated immunity, enhances ability |
| | | of cells to present antigens to T-cells; |
| | | antiviral effects. |
| Monocyte | Peripheral blood | Attracts monocytes to sites of vascular |
| Chemoattractant | monocytes/macrophages | endothelial cell injury, implicated in |
| Protein-1 | | atherosclerosis. |
| (MCP1) | | |
| Colony | | Stimulate the proliferation of specific |
| Stimulating | | pluripotent stem cells of the bone |
| Factors (CSFs) | | marrow in adults. |
| Granulocyte- | | Specific for proliferative effects on |
| CSF (G-CSF) | | cells of the granulocyte lineage; |
| | | proliferative effects on both classes of |
| | | lymphoid cells. |
| Macrophage- | | Specific for cells of the macrophage |
| CSF (M-CSF) | | lineage. |
| Granulocyte- | | Proliferative effects on cells of both |
| MacrophageCSF | | the macrophage and granulocyte |
| (GM-CSF) | | lineages. |

Other cytokines of interest that may be produced by the invention described herein include adhesion molecules (R & D Systems, Adhesion Molecule (1996), at http://www.rndsystems.com); angiogenin (U.S. Pat. No. 4,721,672; Moener et al., 226 Eur. J. Biochem. 483 (1994)); annexin V (Cookson et al., 20 Genomics 463 (1994); Grundmann et al., 85 Proc. Natl. Acad. Sci. USA 3708 (1988); U.S. Pat. No. 5,767,247); caspases (U.S. Pat. No. 6,214,858; Thornberry et al., 281 Science 1312 (1998)); chemokines (U.S. Pat. Nos. 6,174,995; 6,143,289; Sallusto et al., 18 Annu. Rev. Immunol. 593 (2000) Kunkel et al., 59 J. Leukocyte Biol. 81 (1996)); endothelin (U.S. Pat. Nos. 6,242,485; 5,294,569; 5,231,166); eotaxin (U.S. Pat. No. 6,271,347; Ponath et al., 97(3) J. Clin. Invest. 604-612 (1996)); Flt-3 (U.S. Pat. No. 6,190,655); heregulins (U.S. Pat. Nos. 6,284,535; 6,143,740; 6,136,558; 5,859,206; 5,840,525); Leptin (Leroy et al., 271(5) J. Biol. Chem. 2365 (1996); Maffei et al., 92 Proc. Natl. Acad. Sci. USA 6957 (1995); Zhang Y. et al. (1994) Nature 372: 425-432); Macrophage Stimulating Protein (MSP) (U.S. Pat. Nos. 6,248,560; 6,030,949; 5,315,000); Neurotrophic Factors (U.S. Pat. Nos. 6,005,081; 5,288,622); Pleiotrophin/Midkine (PTN/MK) (Pedraza et al., 117 J. Biochem. 845 (1995); Tamura et al., 3 Endocrine 21 (1995); U.S. Pat. No. 5,210,026; Kadomatsu et al., 151 Biochem. Biophys. Res. Commun. 1312 (1988)); STAT proteins (U.S. Pat. Nos. 6,030,808; 6,030,780; Darnell et al., 277 Science 1630-1635 (1997)); Tumor Necrosis Factor Family (Cosman, 7 Blood Cell (1996); Gruss et al., 85 Blood 3378 (1995); Beutler et al., 7 Annu. Rev. Immunol. 625 (1989); Aggarwal et al., 260 J. Biol. Chem. 2345 (1985); Pennica et al., 312 Nature 724 (1984)).

The present invention may also be used to affect blood proteins, a generic name for a vast group of proteins generally circulating in blood plasma, and important for regulating coagulation and clot dissolution. See, e.g., Haematologic Technologies, Inc., HTI Catalog, at www.haemtech.com. Table 3 introduces, in a non-limiting fashion, some of the blood proteins contemplated by the present invention.

TABLE 3
Blood Proteins

| Protein | Principle Activity | Reference |
|---|---|---|
| Factor V | In coagulation, this glycoprotein pro- | Mann et al., 57 ANN. REV. BIOCHEM. |
| | cofactor, is converted to active | 915 (1988); see also Nesheim et al., |
| | cofactor, factor Va, via the serine | 254 J. BIOL. CHEM. 508 (1979); |
| | protease α-thrombin, and less | Tracy et al., 60 BLOOD 59 (1982); |
| | efficiently by its serine protease | Nesheim et al., 80 METHODS |
| | cofactor Xa. The prothrombinase | ENZYMOL. 249 (1981); Jenny et al., |
| | complex rapidly converts zymogen | 84 PROC. NATL. ACAD. SCI. USA |
| | prothrombin to the active serine | 4846 (1987). |
| | protease, α-thrombin. Down | |
| | regulation of prothrombinase | |
| | complex occurs via inactivation of | |
| | Va by activated protein C. | |
| Factor VII | Single chain glycoprotein zymogen | See generally, Broze et al., 80 |
| | in its native form. Proteolytic | METHODS ENZYMOL. 228 (1981); |
| | activation yields enzyme factor VIIa, | Bajaj et al., 256 J. BIOL. CHEM. 253 |
| | which binds to integral membrane | (1981); Williams et al., 264 J. BIOL. |
| | protein tissue factor, forming an | CHEM. 7536 (1989); Kisiel et al., 22 |
| | enzyme complex that proteolytically | THROMBOSIS RES. 375 (1981); |
| | converts factor X to Xa. Also known | Seligsohn et al., 64 J. CLIN. INVEST. |
| | as extrinsic factor Xase complex. | 1056 (1979); Lawson et al., 268 J. |
| | Conversion of VII to VIIa catalyzed | BIOL. CHEM. 767 (1993). |
| | by a number of proteases including | |
| | thrombin, factors IXa, Xa, XIa, and | |
| | XIIa. Rapid activation also occurs | |
| | when VII combines with tissue factor | |
| | in the presence of Ca, likely initiated | |
| | by a small amount of pre-existing | |
| | VIIa. Not readily inhibited by | |
| | antithrombin III/heparin alone, but is | |
| | inhibited when tissue factor added. | |
| Factor IX | Zymogen factor IX, a single chain | Thompson, 67 BLOOD, 565 (1986); |
| | vitamin K-dependent glycoprotein, | Hedner et al., HEMOSTASIS AND |
| | made in liver. Binds to negatively | THROMBOSIS 39-47 (R. W. Colman, J. |
| | charged phospholipid surfaces. | Hirsh, V. J. Marder, E. W. Salzman |
| | Activated by factor XIα or the factor | ed., 2^{nd} ed. J. P. Lippincott Co., |
| | VIIa/tissue factor/phospholipid | Philadelphia) 1987; Fujikawa et al., |
| | complex. Cleavage at one site yields | 45 METHODS IN ENZYMOLOGY 74 |
| | the intermediate IXα, subsequently | (1974). |
| | converted to fully active form IXaβ | |
| | by cleavage at another site. Factor | |
| | IXaβ is the catalytic component of | |
| | the “intrinsic factor Xase complex” | |
| | (factor VIIIa/IXa/Ca²⁺/phospholipid) | |
| | that proteolytically activates factor X | |
| | to factor Xa. | |
| Factor X | Vitamin K-dependent protein | See Davie et al., 48 ADV. ENZYMOL |
| | zymogen, made in liver, circulates in | 277 (1979); Jackson, 49 ANN. REV. |
| | plasma as a two chain molecule | BIOCHEM. 765 (1980); see also |
| | linked by a disulfide bond. Factor Xa | Fujikawa et al., 11 BIOCHEM. 4882 |
| | (activated X) serves as the enzyme | (1972); Discipio et al., 16 BIOCHEM. |
| | component of prothrombinase | 698 (1977); Discipio et al., 18 |
| | complex, responsible for rapid | BIOCHEM. 899 (1979); Jackson et al., |
| | conversion of prothrombin to | 7 BIOCHEM. 4506 (1968); McMullen |
| | thrombin. | et al., 22 BIOCHEM. 2875 (1983). |
| Factor XI | Liver-made glycoprotein homodimer | Thompson et al., 60 J. CLIN. INVEST. |
| | circulates, in a non-covalent complex | 1376 (1977); Kurachi et al., 16 |
| | with high molecular weight | BIOCHEM. 5831 (1977); Bouma et al., |
| | kininogen, as a zymogen, requiring | 252 J. BIOL. CHEM. 6432 (1977); |
| | proteolytic activation to acquire | Wuepper, 31 FED. PROC. 624 (1972); |
| | serine protease activity. Conversion | Saito et al., 50 BLOOD 377 (1977); |
| | of factor XI to factor XIa is catalyzed | Fujikawa et al., 25 BIOCHEM. 2417 |
| | by factor XIIa. XIa unique among | (1986); Kurachi et al., 19 BIOCHEM. |
| | the serine proteases, since it contains | 1330 (1980); Scott et al., 69 J. CLIN. |
| | two active sites per molecule. Works | INVEST. 844 (1982). |
| | in the intrinsic coagulation pathway | |
| | by catalyzing conversion of factor IX | |
| | to factor IXa. Complex form, factor | |
| | XIa/HMWK, activates factor XII to | |
| | factor XIIa and prekallikrein to | |
| | kallikrein. Major inhibitor of XIa is | |
| | a₁-antitrypsin and to lesser extent, | |
| | antithrombin-III. Lack of factor XI | |
| | procoagulant activity causes bleeding | |
| | disorder: plasma thromboplastin | |
| | antecedent deficiency. | |
| Factor XII | Glycoprotein zymogen. Reciprocal | Schmaier et al., 18-38, and Davie, |
| (Hageman | activation of XII to active serine | 242-267 HEMOSTASIS & |
| Factor) | protease factor XIIa by kallikrein is | THROMBOSIS (Colman et al., eds., |
| | central to start of intrinsic | J. B. Lippincott Co., Philadelphia, |
| | coagulation pathway. Surface bound | 1987). |
| | α-XIIa activates factor XI to XIa. | |
| | Secondary cleavage of α-XIIa by | |
| | kallikrein yields β-XIIa, and | |
| | catalyzes solution phase activation of | |
| | kallikrein, factor VII and the | |
| | classical complement cascade. | |
| Factor XIII | Zymogenic form of glutaminyl- | See McDonaugh, 340-357 |
| | peptide γ-glutamyl transferase factor | HEMOSTASIS & THROMBOSIS |
| | XIIIa (fibrinoligase, plasma | (Colman et al., eds., J. B. Lippincott |
| | transglutaminase, fibrin stabilizing | Co., Philadelphia, 1987); Folk et al., |
| | factor). Made in the liver, found | 113 METHODS ENZYMOL. 364 |
| | extracellularly in plasma and | (1985); Greenberg et al., 69 BLOOD |
| | intracellularly in platelets, | 867 (1987). Other proteins known to |
| | megakaryocytes, monocytes, | be substrates for Factor XIIIa, that |
| | placenta, uterus, liver and prostrate | may be hemostatically important, |
| | tissues. Circulates as a tetramer of 2 | include fibronectin (Iwanaga et al., |
| | pairs of nonidentical subunits (A₂B₂). | 312 ANN. NY ACAD. SCI. 56 (1978)), |
| | Full expression of activity is | a₂-antiplasmin (Sakata et al., 65 J. CLIN. |
| | achieved only after the Ca²⁺-and | INVEST. 290 (1980)), collagen |
| | fibrin(ogen)-dependent dissociation | (Mosher et al., 64 J. CLIN. INVEST. |
| | of B subunit dimer from A₂′ dimer. | 781 (1979)), factor V (Francis et al., |
| | Last of the zymogens to become | 261 J. BIOL. CHEM. 9787 (1986)), |
| | activated in the coagulation cascade, | von Willebrand Factor (Mosher et |
| | the only enzyme in this system that is | al., 64 J. CLIN. INVEST. 781 (1979)) |
| | not a serine protease. XIIIa stabilizes | and thrombospondin (Bale et al., 260 |
| | the fibrin clot by crosslinking the α | J. BIOL. CHEM. 7502 (1985); Bohn, |
| | and γ-chains of fibrin. Serves in cell | 20 MOL. CELL BIOCHEM. 67 (1978)). |
| | proliferation in wound healing, tissue | |
| | remodeling, atherosclerosis, and | |
| | tumor growth. | |
| Fibrinogen | Plasma fibrinogen, a large | FURLAN, Fibrinogen, IN HUMAN |
| | glycoprotein, disulfide linked dimer | PROTEIN DATA, (Haeberli, ed., VCH |
| | made of 3 pairs of non-identical | Publishers, N.Y.,1995); Doolittle, in |
| | chains (Aa, Bb and g), made in liver. | HAEMOSTASIS & THROMBOSIS, 491-513 |
| | Aa has N-terminal peptide | (3rd ed., Bloom et al., eds., |
| | (fibrinopeptide A (FPA), factor XIIIa | Churchill Livingstone, 1994); |
| | crosslinking sites, and 2 | HANTGAN, et al., in HAEMOSTASIS & |
| | phosphorylation sites. Bb has | THROMBOSIS 269-89 (2d ed., Forbes |
| | fibrinopeptide B (FPB), 1 of 3 N- | et al., eds., Churchill Livingstone, |
| | linked carbohydrate moieties, and an | 1991). |
| | N-terminal pyroglutamic acid. The g | |
| | chain contains the other N-linked | |
| | glycos. site, and factor XIIIa cross- | |
| | linking sites. Two elongated subunits | |
| | ((AaBbg)₂) align in an antiparallel | |
| | way forming a trinodular | |
| | arrangement of the 6 chains. Nodes | |
| | formed by disulfide rings between | |
| | the 3 parallel chains. Central node | |
| | (n-disulfide knot, E domain) formed | |
| | by N-termini of all 6 chains held | |
| | together by 11 disulfide bonds, | |
| | contains the 2 IIa-sensitive sites. | |
| | Release of FPA by cleavage | |
| | generates Fbn I, exposing a | |
| | polymerization site on Aa chain. | |
| | These sites bind to regions on the D | |
| | domain of Fbn to form proto-fibrils. | |
| | Subsequent IIa cleavage of FPB from | |
| | the Bb chain exposes additional | |
| | polymerization sites, promoting | |
| | lateral growth of Fbn network. Each | |
| | of the 2 domains between the central | |
| | node and the C-terminal nodes | |
| | (domains D and E) has parallel ahelical | |
| | regions of the Aa, Bb and g | |
| | chains having protease-(plasmin-) | |
| | sensitive sites. Another major | |
| | plasmin sensitive site is in | |
| | hydrophilic preturbance of a-chain | |
| | from C-terminal node. Controlled | |
| | plasmin degradation converts Fbg | |
| | into fragments D and E. | |
| Fibronectin | High molecular weight, adhesive, | Skorstengaard et al., 161 Eur. J. |
| | glycoprotein found in plasma and | BIOCHEM. 441 (1986); Kornblihtt et |
| | extracellular matrix in slightly | al., 4 EMBO J. 1755 (1985); |
| | different forms. Two peptide chains | Odermatt et al., 82 PNAS 6571 |
| | interconnected by 2 disulfide bonds, | (1985); Hynes, R. O., ANN. REV. |
| | has 3 different types of repeating | CELL BIOL., 1, 67 (1985); Mosher 35 |
| | homologous sequence units. | ANN. REV. MED. 561 (1984); |
| | Mediates cell attachment by | Rouslahti et al., 44 Cell 517 (1986); |
| | interacting with cell surface | Hynes 48 CELL 549 (1987); Mosher |
| | receptors and extracellular matrix | 250 BIOL. CHEM. 6614 (1975). |
| | components. Contains an Arg-Gly- | |
| | Asp-Ser (RGDS) cell attachment- | |
| | promoting sequence, recognized by | |
| | specific cell receptors, such as those | |
| | on platelets. Fibrin-fibronectin | |
| | complexes stabilized by factor XIIIa- | |
| | catalyzed covalent cross-linking of | |
| | fibronectin to the fibrin a chain. | |
| b₂- | Also called b₂I and Apolipoprotein | See, e.g., Lozier et al., 81 PNAS |
| Glycoprotein I | H. Highly glycosylated single chain | 2640-44 (1984); Kato & Enjyoi 30 |
| | protein made in liver. Five repeating | BIOCHEM. 11687-94 (1997); Wurm, |
| | mutually homologous domains | 16 INT'L J. BIOCHEM. 511-15 (1984); |
| | consisting of approximately 60 | Bendixen et al., 31 BIOCHEM. 3611-17 |
| | amino acids disulfide bonded to form | (1992); Steinkasserer et al., 277 |
| | Short Consensus Repeats (SCR) or | BIOCHEM. J. 387-91 (1991); Nimpf |
| | Sushi domains. Associated with | et al., 884 BIOCHEM. BIOPHYS. ACTA |
| | lipoproteins, binds anionic surfaces | 142-49 (1986); Kroll et. al. 434 |
| | like anionic vesicles, platelets, DNA, | BIOCHEM. BIOPHYS. Acta 490-501 |
| | mitochondria, and heparin. Binding | (1986); Polz et al., 11 INT'L J. |
| | can inhibit contact activation | BIOCHEM. 265-73 (1976); McNeil et |
| | pathway in blood coagulation. | al., 87 PNAS 4120-24 (1990); Galli |
| | Binding to activated platelets inhibits | et al;. I LANCET 1544-47 (1990); |
| | platelet associated prothrombinase | Matsuuna et al., II LANCET 177-78 |
| | and adenylate cyclase activities. | (1990); Pengo et al., 73 THROMBOSIS |
| | Complexes between b₂I and | & HAEMOSTASIS 29-34 (1995). |
| | cardiolipin have been implicated in | |
| | the anti-phospholipid related immune | |
| | disorders LAC and SLE. | |
| Osteonectin | Acidic, noncollagenous glycoprotein | Villarreal et al., 28 BIOCHEM. 6483 |
| | (Mr = 29,000) originally isolated from | (1989); Tracy et al., 29 INT'L J. |
| | fetal and adult bovine bone matrix. | BIOCHEM. 653 (1988); Romberg et |
| | May regulate bone metabolism by | al., 25 BIOCHEM. 1176 (1986); Sage |
| | binding hydroxyapatite to collagen. | & Bornstein 266 J. BIOL. CHEM. |
| | Identical to human placental SPARC. | 14831 (1991); Kelm & Mann 4 J. |
| | An alpha granule component of | BONE MIN. RES. 5245 (1989); Kelm |
| | human platelets secreted during | et al., 80 BLOOD 3112 (1992). |
| | activation. A small portion of | |
| | secreted osteonectin expressed on the | |
| | platelet cell surface in an activation- | |
| | dependent manner | |
| Plasminogen | Single chain glycoprotein zymogen | See Robbins, 45 METHODS IN |
| | with 24 disulfide bridges, no free | ENZYMOLOGY 257 (1976); COLLEN, |
| | sulfhydryls, and 5 regions of internal | 243-258 BLOOD COAG. (Zwaal et al., |
| | sequence homology, “kringles”, each | eds., New York, Elsevier, 1986); see |
| | five triple-looped, three disulfide | also Castellino et al., 80 METHODS IN |
| | bridged, and homologous to kringle | ENZYMOLOGY 365 (1981); Wohl et |
| | domains in t-PA, u-PA and | al., 27 THROMB. RES. 523 (1982); |
| | prothrombin. Interaction of | Barlow et al., 23 BIOCHEM. 2384 |
| | plasminogen with fibrin and α2- | (1984); SOTTRUP-JENSEN ET AL., 3 |
| | antiplasmin is mediated by lysine | PROGRESS IN CHEM. FIBRINOLYSIS & |
| | binding sites. Conversion of | THROMBOLYSIS 197-228 (Davidson |
| | plasminogen to plasmin occurs by | et al., eds., Raven Press, New York |
| | variety of mechanisms, including | 1975). |
| | urinary type and tissue type | |
| | plasminogen activators, | |
| | streptokinase, staphylokinase, | |
| | kallikrein, factors IXa and XIIa, but | |
| | all result in hydrolysis at Arg560-Val561, | |
| | yielding two chains that | |
| | remain covalently associated by a | |
| | disulfide bond. | |
| tissue | t-PA, a serine endopeptidase | See Plasminogen. |
| Plasminogen | synthesized by endothelial cells, is | |
| Activator | the major physiologic activator of | |
| | plasminogen in clots, catalyzing | |
| | conversion of plasminogen to | |
| | plasmin by hydrolising a specific | |
| | arginine-alanine bond. Requires | |
| | fibrin for this activity, unlike the | |
| | kidney-produced version, urokinase- | |
| | PA. | |
| Plasmin | See Plasminogen. Plasmin, a serine | See Plasminogen. |
| | protease, cleaves fibrin, and activates | |
| | and/or degrades compounds of | |
| | coagulation, kinin generation, and | |
| | complement systems. Inhibited by a | |
| | number of plasma protease inhibitors | |
| | in vitro. Regulation of plasmin in | |
| | vivo occurs mainly through | |
| | interaction with a₂-antiplasmin, and | |
| | to a lesser extent, a₂-macroglobulin. | |
| Platelet Factor-4 | Low molecular weight, heparin- | Rucinski et al., 53 BLOOD 47 (1979); |
| | binding protein secreted from | Kaplan et al., 53 BLOOD 604 (1979); |
| | agonist-activated platelets as a | George 76 BLOOD 859 (1990); Busch |
| | homotetramer in complex with a | et al., 19 THROMB. RES. 129 (1980); |
| | high molecular weight, proteoglycan, | Rao et al., 61 BLOOD 1208 (1983); |
| | carrier protein. Lysine-rich, COOH- | Brindley, et al., 72 J. CLIN. INVEST. |
| | terminal region interacts with cell | 1218 (1983); Deuel et al., 74 PNAS |
| | surface expressed heparin-like | 2256 (1981); Osterman et al., 107 |
| | glycosaminoglycans on endothelial | BIOCHEM. BIOPHYS. RES. COMMUN. |
| | cells, PF-4 neutralizes anticoagulant | 130 (1982); Capitanio et al., 839 |
| | activity of heparin exerts | BIOCHEM. BIOPHYS. ACTA 161 |
| | procoagulant effect, and stimulates | (1985). |
| | release of histamine from basophils. | |
| | Chemotactic activity toward | |
| | neutrophils and monocytes. Binding | |
| | sites on the platelet surface have | |
| | been identified and may be important | |
| | for platelet aggregation. | |
| Protein C | Vitamin K-dependent zymogen, | See Esmon, 10 PROGRESS IN |
| | protein C, made in liver as a single | THROMB. & HEMOSTS. 25 (1984); |
| | chain polypeptide then converted to a | Stenflo, 10 SEMIN. IN THROMB. & |
| | disulfide linked heterodimer. | HEMOSTAS. 109 (1984); Griffen et |
| | Cleaving the heavy chain of human | al., 60 BLOOD 261 (1982); Kisiel et |
| | protein C converts the zymogen into | al., 80 METHODS ENZYMOL. 320 |
| | the serine protease, activated protein | (1981); Discipio et al., 18 BIOCHEM. |
| | C. Cleavage catalyzed by a complex | 899 (1979). |
| | of α-thrombin and thrombomodulin. | |
| | Unlike other vitamin K dependent | |
| | coagulation factors, activated protein | |
| | C is an anticoagulant that catalyzes | |
| | the proteolytic inactivation of factors | |
| | Va and VIIIa, and contributes to the | |
| | fibrinolytic response by complex | |
| | formation with plasminogen | |
| | activator inhibitors. | |
| Protein S | Single chain vitamin K-dependent | Walker 10 SEMIN. THROMB. |
| | protein functions in coagulation and | HEMOSTAS. 131 (1984); Dahlback et |
| | complement cascades. Does not | al., 10 SEMIN. THROMB. HEMOSTAS., |
| | possess the catalytic triad. | 139 (1984); Walker 261 J. BIOL. |
| | Complexes to C4b binding protein | CHEM. 10941 (1986). |
| | (C4BP) and to negatively charged | |
| | phospholipids, concentrating C4BP | |
| | at cell surfaces following injury. | |
| | Unbound S serves as anticoagulant | |
| | cofactor protein with activated | |
| | Protein C. A single cleavage by | |
| | thrombin abolishes protein S | |
| | cofactor activity by removing gla | |
| | domain. | |
| Protein Z | Vitamin K-dependent, single-chain | Sejima et al., 171 BIOCHEM. |
| | protein made in the liver. Direct | BIOPHYSICS RES. COMM. 661 (1990); |
| | requirement for the binding of | Hogg et al., 266 J. BIOL. CHEM. |
| | thrombin to endothelial | 10953 (1991); Hogg et al., 17 |
| | phospholipids. Domain structure | BIOCHEM. BIOPHYSICS RES. COMM. |
| | similar to that of other vitamin K- | 801 (1991); Han et al., 38 BIOCHEM. |
| | dependant zymogens like factors VII, | 11073 (1999); Kemkes-Matthes et |
| | IX, X, and protein C. N-terminal | al., 79 THROMB. RES. 49 (1995). |
| | region contains carboxyglutamic acid | |
| | domain enabling phospholipid | |
| | membrane binding. C-terminal | |
| | region lacks “typical” serine protease | |
| | activation site. Cofactor for | |
| | inhibition of coagulation factor Xa | |
| | by serpin called protein Z-dependant | |
| | protease inhibitor. Patients diagnosed | |
| | with protein Z deficiency have | |
| | abnormal bleeding diathesis during | |
| | and after surgical events. | |
| Prothrombin | Vitamin K-dependent, single-chain | Mann et al., 45 METHODS IN |
| | protein made in the liver. Binds to | ENZYMOLOGY 156 (1976); |
| | negatively charged phospholipid | Magnusson et al., PROTEASES IN |
| | membranes. Contains two “kringle” | BIOLOGICAL CONTROL 123-149 |
| | structures. Mature protein circulates | (Reich et al., eds. Cold Spring |
| | in plasma as a zymogen and, during | Harbor Labs., New York 1975); |
| | coagulation, is proteolytically | Discipio et al., 18 BIOCHEM. 899 |
| | activated to the potent serine | (1979). |
| | protease α-thrombin. | |
| α-Thrombin | See Prothrombin. During | 45 METHODS ENZYMOL. 156 (1976). |
| | coagulation, thrombin cleaves | |
| | fibrinogen to form fibrin, the | |
| | terminal proteolytic step in | |
| | coagulation, forming the fibrin clot. | |
| | Thrombin also responsible for | |
| | feedback activation of procofactors | |
| | V and VIII. Activates factor XIII and | |
| | platelets, functions as vasoconstrictor | |
| | protein. Procoagulant activity | |
| | arrested by heparin cofactor II or the | |
| | antithrombin III/heparin complex, or | |
| | complex formation with | |
| | thrombomodulin. Formation of | |
| | thrombin/thrombomodulin complex | |
| | results in inability of thrombin to | |
| | cleave fibrinogen and activate factors | |
| | V and VIII, but increases the | |
| | efficiency of thrombin for activation | |
| | of the anticoagulant, protein C. | |
| b-Thrombo- | Low molecular weight, heparin- | See, e.g., George 76 BLOOD 859 |
| globulin | binding, platelet-derived tetramer | (1990); Holt & Niewiarowski 632 |
| | protein, consisting of four identical | BIOCHEM. BIOPHYS. ACTA 284 |
| | peptide chains. Lower affinity for | (1980); Niewiarowski et al., 55 |
| | heparin than PF-4. Chemotactic | BLOOD 453 (1980); Varma et al., 701 |
| | activity for human fibroblasts, other | BIOCHEM. BIOPHYS. ACTA 7 (1982); |
| | functions unknown. | Senior et al., 96 J. CELL. BIOL. 382 |
| | | (1983). |
| Thrombopoietin | Human TPO (Thrombopoietin, Mpl- | Horikawa et al., 90(10) BLOOD 4031-38 |
| | ligand, MGDF) stimulates the | (1997); de Sauvage et al., 369 |
| | proliferation and maturation of | NATURE 533-58 (1995). |
| | megakaryocytes and promotes | |
| | increased circulating levels of | |
| | platelets in vivo. Binds to c-Mpl | |
| | receptor. | |
| Thrombo- | High-molecular weight, heparin- | Dawes et al., 29 THROMB. RES. 569 |
| spondin | binding glycoprotein constituent of | (1983); Switalska et al., 106 J. LAB. |
| | platelets, consisting of three, | CLIN. MED. 690 (1985); Lawler et al. |
| | identical, disulfide-linked | 260 J. BIOL. CHEM. 3762 (1985); |
| | polypeptide chains. Binds to surface | Wolff et al., 261 J. BIOL. CHEM. 6840 |
| | of resting and activated platelets, | (1986); Asch et al., 79 J. CLIN. |
| | may effect platelet adherence and | CHEM. 1054 (1987); Jaffe et al., 295 |
| | aggregation. An integral component | NATURE 246 (1982); Wright et al., |
| | of basement membrane in different | 33 J. HISTOCHEM. CYTOCHEM. 295 |
| | tissues. Interacts with a variety of | (1985); Dixit et al., 259 J. BIOL. |
| | extracellular macromolecules | CHEM. 10100 (1984); Mumby et al., |
| | including heparin, collagen, | 98 J. CELL. BIOL. 646 (1984); Lahav |
| | fibrinogen and fibronectin, | et al, 145 EUR. J. BIOCHEM. 151 |
| | plasminogen, plasminogen activator, | (1984); Silverstein et al, 260 J. BIOL. |
| | and osteonectin. May modulate cell- | CHEM. 10346 (1985); Clezardin et al. |
| | matrix interactions. | 175 EUR. J. BIOCHEM. 275 (1988); |
| | | Sage & Bornstein (1991). |
| Von Willebrand | Multimeric plasma glycoprotein | Hoyer 58 BLOOD 1 (1981); Ruggeri |
| Factor | made of identical subunits held | & Zimmerman 65 J. CLIN. INVEST. |
| | together by disulfide bonds. During | 1318 (1980); Hoyer & Shainoff 55 |
| | normal hemostasis, larger multimers | BLOOD 1056 (1980); Meyer et al., 95 |
| | of vWF cause platelet plug formation | J. LAB. CLIN. INVEST. 590 (1980); |
| | by forming a bridge between platelet | Santoro 21 THROMB. RES. 689 |
| | glycoprotein IB and exposed | (1981); Santoro, & Cowan 2 |
| | collagen in the subendothelium. Also | COLLAGEN RELAT. RES. 31 (1982); |
| | binds and transports factor VIII | Morton et al., 32 THROMB. RES. 545 |
| | (antihemophilic factor) in plasma. | (1983); Tuddenham et al., 52 BRIT. J. HAEMATOL. |
| | | 259 (1982). |

Additional blood proteins contemplated herein include the following human serum proteins, which may also be placed in another category of protein (such as hormone or antigen): Actin, Actinin, Amyloid Serum P, Apolipoprotein E, B2-Microglobulin, C-Reactive Protein (CRP), Cholesterylester transfer protein (CETP), Complement C3B, Ceruplasmin, Creatine Kinase, Cystatin, Cytokeratin 8, Cytokeratin 14, Cytokeratin 18, Cytokeratin 19, Cytokeratin 20, Desmin, Desmocollin 3, FAS (CD95), Fatty Acid Binding Protein, Ferritin, Filamin, Glial Filament Acidic Protein, Glycogen Phosphorylase Isoenzyme BB (GPBB), Haptoglobulin, Human Myoglobin, Myelin Basic Protein, Neurofilament, Placental Lactogen, Human SHBG, Human Thyroid Peroxidase, Receptor Associated Protein, Human Cardiac Troponin C, Human Cardiac Troponin I, Human Cardiac Troponin T, Human Skeletal Troponin I, Human Skeletal Troponin T, Vimentin, Vinculin, Transferrin Receptor, Prealbumin, Albumin, Alpha-1-Acid Glycoprotein, Alpha-1-Antichymotrypsin, Alpha-1-Antitrypsin, Alpha-Fetoprotein, Alpha-1-Microglobulin, Beta-2-microglobulin, C-Reactive Protein, Haptoglobulin, Myoglobulin, Prealbumin, PSA, Prostatic Acid Phosphatase, Retinol Binding Protein, Thyroglobulin, Thyroid Microsomal Antigen, Thyroxine Binding Globulin, Transferrin, Troponin I, Troponin T, Prostatic Acid Phosphatase, Retinol Binding Globulin (RBP). All of these proteins, and sources thereof, are known in the art. Many of these proteins are available commercially from, for example, Research Diagnostics, Inc. (Flanders, N.J.).

The cell clone of the present invention may also express neurotransmitters, or functional portions thereof. Neurotransmitters are chemicals made by neurons and used by them to transmit signals to the other neurons or non-neuronal cells (e.g., skeletal muscle; myocardium, pineal glandular cells) that they innervate. Neurotransmitters produce their effects by being released into synapses when their neuron of origin fires (i.e., becomes depolarized) and then attaching to receptors in the membrane of the post-synaptic cells. This causes changes in the fluxes of particular ions across that membrane, making cells more likely to become depolarized, if the neurotransmitter happens to be excitatory, or less likely if it is inhibitory. Neurotransmitters can also produce their effects by modulating the production of other signal-transducing molecules (“second messengers”) in the post-synaptic cells. See, e.g., Cooper, Bloom & Roth, The Biochemical Basis of Neuropharmacology (7th Ed. Oxford Univ. Press, NYC, 1996); http://web.indstate.edu/thcme/mwking/nerves. Neurotransmitters contemplated in the present invention include, but are not limited to, Acetylcholine, Serotonin, γ-aminobutyrate (GABA), Glutamate, Aspartate, Glycine, Histamine, Epinephrine, Norepinephrine, Dopamine, Adenosine, ATP, Nitric oxide, and any of the peptide neurotransmitters such as those derived from pre-opiomelanocortin (POMC), as well as antagonists and agonists of any of the foregoing.

Numerous other proteins or peptides may serve as either targets, or as a source of target-binding moieties as described herein. Table 4 presents a non-limiting list and description of some pharmacologically active peptides that may serve as, or serve as a source of a functional derivative of, the target of the present invention.

TABLE 4
Pharmacologically active peptides

| Binding partner/ | | |
|---|---|---|
| Protein of interest | | |
| (form of peptide) | Pharmacological activity | Reference |
| EPO receptor | EPO mimetic | Wrighton et al., 273 SCIENCE 458-63 |
| (intrapeptide | | (1996); U.S. Pat. No. 5,773,569, |
| disulfide-bonded) | | issued Jun. 30, 1998. |
| EPO receptor | EPO mimetic | Livnah et al., 273 SCIENCE 464-71 |
| (C-terminally cross- | | (1996); Wrighton et al., 15 NATURE |
| linked dimer) | | BIOTECHNOLOGY 1261-5 (1997); Int'l |
| | | Patent Application WO 96/40772, |
| | | published Dec. 19, 1996. |
| EPO receptor | EPO mimetic | Naranda et al., 96 PNAS 7569-74 |
| (linear) | | (1999). |
| c-Mpl | TPO-mimetic | Cwirla et al., 276 SCIENCE 1696-9 |
| (linear) | | (1997); U.S. Pat. No. 5,869,451, |
| | | issued Feb. 9, 1999; U.S. Pat. No. |
| | | 5,932,946, issued Aug. 3, 1999. |
| c-Mpl | TPO-mimetic | Cwirla et al., 276 SCIENCE 1696-9 |
| (C-terminally cross- | | (1997). |
| linked dimer) | | |
| (disulfide-linked | stimulation of | Paukovits et al., 364 HOPPE-SEYLERS |
| dimer) | hematopoesis | Z. PHYSIOL. CHEM. 30311 (1984); |
| | (“G-CSF- | Laerurngal., 16 EXP. HEMAT. 274-80 |
| | mimetic”) | (1988). |
| (alkylene-linked dimer) | G-CSF-mimetic | Batnagar et al., 39 J. MED. CHEM. |
| | | 38149 (1996); Cuthbertson et al., 40 J. |
| | | MED. CHEM. 2876-82 (1997); King et |
| | | al., 19 EXP. HEMATOL. 481 (1991); |
| | | King et al., 86 (Suppl. 1) BLOOD 309 |
| | | (1995). |
| IL-1 receptor | inflammatory and | U.S. Pat. No. 5,608,035; U.S. Pat. No. |
| (linear) | autoimmune diseases | 5,786,331; U.S Pat. No. 5,880,096; |
| | (“IL-1 antagonist” or “IL- | Yanofsky et al., 93 PNAS 7381-6 |
| | 1 ra-mimetic”) | (1996); Akeson et al., 271 J. BIOL. |
| | | CHEM. 30517-23 (1996); Wiekzorek et |
| | | al., 49 POL. J. PHARMACOL. 107-17 |
| | | (1997); Yanofsky, 93 PNAS 7381-7386 |
| | | (1996). |
| Facteur thyrnique | stimulation of | Inagaki-Ohara et al., 171 CELLULAR |
| (linear) | lymphocytes (FTS- | IMMUNOL. 30-40 (1996); Yoshida, 6 |
| | mimetic) | J. IMMUNOPHARMACOL 141-6 (1984). |
| CTLA4 MAb | CTLA4-mimetic | Fukumoto et al., 16 NATURE BIOTECH. |
| (intrapeptide di-sulfide | | 267-70 (1998). |
| bonded) | | |
| TNF-a receptor | TNF-a antagonist | Takasaki et al., 15 NATURE BIOTECH. |
| (exo-cyclic) | | 1266-70 (1997); WO 98/53842, |
| | | published Dec. 3, 1998. |
| TNF-a receptor | TNF-a antagonist | Chirinos-Rojas, J. IMM., 5621-26. |
| (linear) | | |
| C3b | inhibition of complement | Sahu et al., 157 IMMUNOL. 884-91 |
| (intrapeptide di-sulfide | activation; autoimmune | (1996); Morikis et al., 7 PROTEIN SCI. |
| bonded) | diseases (C3b antagonist) | 619-27 (1998). |
| vinculin | cell adhesion processes, | Adey et al., 324 BIOCHEM. J. 523-8 |
| (linear) | cell growth, differentiation | (1997). |
| | wound healing, tumor | |
| | metastasis (“vinculin | |
| | binding”) | |
| C4 binding protein (C413P) | anti-thrombotic | Linse et al. 272 BIOL. CHEM. 14658-65 |
| (linear) | | (1997). |
| urokinase receptor | processes associated with | Goodson et al., 91 PNAS 7129-33 |
| (linear) | urokinase interaction with | (1994); International patent |
| | its receptor (e.g. | application WO 97/35969, published |
| | angiogenesis, tumor cell | Oct. 2, 1997. |
| | invasion and metastasis; | |
| | (URK antagonist) | |
| Mdm2, Hdm2 | Inhibition of inactivation | Picksley et al., 9 ONCOGENE 2523-9 |
| (linear) | of p53 mediated by Mdm2 | (1994); Bottger et al. 269 J. MOL. |
| | or hdm2; anti-tumor | BIOL. 744-56 (1997); Bottger et al., 13 |
| | (“Mdm/hdm antagonist”) | ONCOGENE 13: 2141-7 (1996). |
| p21^{WAF1} | anti-tumor by mimicking | Ball et al., 7 CURR. BIOL. 71-80 |
| (linear) | the activity of p21^{WAF1} | (1997). |
| farnesyl transferase | anti-cancer by preventing | Gibbs et al., 77 CELL 175-178 (1994). |
| (linear) | activation of ras oncogene | |
| Ras effector domain | anti-cancer by inhibiting | Moodie et at., 10 TRENDS GENEL 44-48 |
| (linear) | biological function of the | (1994); Rodriguez et al., 370 |
| | ras oncogene | NATURE 527-532 (1994). |
| SH2/SH3 domains | anti-cancer by inhibiting | Pawson et al, 3 CURR. BIOL. 434-432 |
| (linear) | tumor growth with | (1993); Yu et al., 76 CELL 933-945 |
| | activated tyrosine kinases | (1994). |
| p16^{INK4} | anti-cancer by mimicking | Fahraeus et al., 6 CURR. BIOL. 84-91 |
| (linear) | activity of p16; e.g., | (1996). |
| | inhibiting cyclin D-Cdk | |
| | complex (“p, 16-mimetic”) | |
| Src, Lyn | inhibition of Mast cell | Stauffer et al., 36 BIOCHEM. 9388-94 |
| (linear) | activation, IgE-related | (1997). |
| | conditions, type I | |
| | hypersensitivity (“Mast | |
| | cell antagonist”). | |
| Mast cell protease | treatment of inflammatory | International patent application WO |
| (linear) | disorders mediated by | 98/33812, published Aug. 6, 1998. |
| | release of tryptase-6 | |
| | (“Mast cell protease | |
| | inhibitors”) | |
| SH3 domains | treatment of SH3- | Rickles et al., 13 EMBO J. |
| (linear) | mediated disease states | 5598-5604 (1994); Sparks et |
| | (“SH3 antagonist”) | al., 269 J. BIOL. CHEM. |
| | | 238536 (1994); Sparks et al., |
| | | 93 PNAS 1540-44 (1996). |
| HBV core antigen (HBcAg) | treatment of HBV viral | Dyson & Muray, PNAS 2194-98 |
| (linear) | antigen (HBcAg) | (1995). |
| | infections (“anti-HBV”) | |
| selectins | neutrophil adhesion | Martens et al., 270 J. BIOL. |
| (linear) | inflammatory diseases | CHEM. 21129-36 (1995); |
| | (“selectin antagonist”) | European Pat. App. EP 0 714 |
| | | 912, published Jun. 5, 1996. |
| calmodulin | calmodulin | Pierce et al., 1 MOLEC. |
| (linear, cyclized) | antagonist | DIVEMILY 25965 (1995); |
| | | Dedman et al., 267 J. BIOL. |
| | | CHEM. 23025-30 (1993); |
| | | Adey & Kay, 169 GENE 133-34 |
| | | (1996). |
| integrins | tumor-homing; treatment | International patent applications WO |
| (linear, cyclized) | for conditions related to | 95/14714, published Jun. 1, 1995; |
| | integrin-mediated cellular | WO 97/08203, published Mar. |
| | events, including platelet | 6, 1997; WO 98/10795, published |
| | aggregation, thrombosis, | Mar. 19, 1998; WO 99/24462, |
| | wound healing, | published May 20, 1999; Kraft et al., |
| | osteoporosis, tissue repair, | 274 J. BIOL. CHEM. 1979-85 (1999). |
| | angiogenesis (e.g., for | |
| | treatment of cancer) and | |
| | tumor invasion (“integrin- | |
| | binding”) | |
| fibronectin and | treatment of inflammatory | International patent application WO |
| extracellular matrix | and autoimmune | 98/09985, published March 12, 1998. |
| components of T-cells and | conditions | |
| macrophages | | |
| (cyclic, linear) | | |
| somatostatin and cortistatin | treatment or prevention of | European patent application EP 0 911 |
| (linear) | hormone-producing | 393, published Apr. 28, 1999. |
| | tumors, acromegaly, | |
| | giantism, dementia, gastriculcer, | |
| | tumor growth, | |
| | inhibition of hormone | |
| | secretion, modulation of | |
| | sleep or neural activity | |
| bacterial lipopoly-saccharide | antibiotic; septic shock; | U.S. Pat. No. 5,877,151, issued Mar. |
| (linear) | disorders modulatable by | 2, 1999. |
| | CAP37 | |
| parclaxin, mellitin | antipathogenic | International patent application WO |
| (linear or cyclic) | | 97/31019, published 28 Aug. 1997. |
| VIP | impotence, neuro- | International patent application WO |
| (linear, cyclic) | degenerative disorders | 97/40070, published Oct. 30, |
| | | 1997. |
| CTLs | cancer | European patent application EP 0 770 |
| (linear) | | 624, published May 2, 1997. |
| THF-gamma2 | | Burnstein, 27 BIOCHEM. 4066-71 |
| (linear) | | (1988). |
| Amylin | | Cooper, 84 PNAS 8628-32 (1987). |
| (linear) | | |
| Adreno-medullin | | Kitamura, 192 BBRC 553-60 (1993). |
| (linear) | | |
| VEGF | anti-angiogenic; cancer, | Fairbrother, 37 BIOCHEM. 17754-64 |
| (cyclic, linear) | rheumatoid arthritis, | (1998). |
| | diabetic retinopathy, | |
| | psoriasis (“VEGF | |
| | antagonist’”) | |
| MMP | inflammation and | Koivunen, 17 NATURE BIOTECH. 768-74 |
| (cyclic) | autoimmune disorders; | (1999). |
| | tumor growth (“MMP | |
| | inhibitor”) | |
| HGH fragment | | U.S. Pat. No. 5,869,452, |
| (linear) | | issued Feb. 9, 1999. |
| Echistatin | inhibition of platelet | Gan, 263 J. BIOL. 19827-32 (1988). |
| | aggregation | |
| SLE autoantibody | SLE | International patent application WO |
| (linear) | | 96/30057, published Oct. 3, 1996. |
| GD1 alpha | suppression of tumor | Ishikawa et al., 1 FEBS LETT. 20-4 |
| | metastasis | (1998). |
| anti-phospholipid β-2 | endothelial cell activation, | Blank Mal., 96 PNAS 5164-8 (1999). |
| glycoprotein-1 (β2GPI) | anti-phospholipid | |
| antibodies | syndrome (APS), | |
| | thromboembolic | |
| | phenomena, | |
| | thrombocytopenia, and | |
| | recurrent fetal loss | |
| T-Cell Receptor β chain | diabetes | International patent application WO |
| (linear) | | 96/101214, published Apr. 18, 1996. |
| EPO receptor | EPO mimetic | Wrighton et al. (1996), Science 273: |
| (intrapeptide | | 458-63; U.S. Pat. No. 5,773,569, |
| disulfide-bonded) | | issued Jun. 30, 1998 to Wrighton et |
| | | al. |
| EPO receptor | EPO mimetic | Livnah et al. (1996), Science 273: |
| (C-terminally cross- | | 464-71; Wrighton et al. (1997), |
| linked dimer) | | Nature Biotechnology 15: 1261-5; |
| | | int'1 patent application WO |
| | | 96/40772, published Dec. 19, 1996 |
| EPO receptor | EPO mimetic | Naranda et al., 96 PNAS 7569-74 |
| (linear) | | (1999) |
| c-Mpl | TPO-mimetic | Cwirla et al. (1997) Science 276: 1696-9; |
| (linear) | | U.S. Pat. No. 5,869,451, issued Feb. |
| | | 9, 1999; U.S. Pat. No. 5,932,946, |
| | | issued Aug. 3, 1999 |
| c-Mpl | TPO-mimetic | Cwirla et al. (1997) Science 276: 1696-9 |
| (C-terminally cross- | | |
| linked dimer) | | |
| (disulfide-linked | stimulation of | Paukovits et al. (1984), Hoppe- |
| dimer) | hematopoesis | Seylers Z. Physiol. Chem. 365: |
| | (“G-CSF- | 30311; Laerurn gal. (1988), Exp. |
| | mimetic”) | Hemat. 16: 274-80 |
| (alkylene-linked dimer) | G-CSF-mimetic | Batnagar 91-al. (1996), linked dimer J. |
| | | Med. Chem. 39: 38149; Cuthbertson et |
| | | al. (1997), J. Med. Chem. 40: 2876-82; |
| | | King et al. (1991), Exp. Hematol. |
| | | 19: 481; King et al. (1995), Blood 86 |
| | | (Suppl. 1): 309 |
| IL-1 receptor | inflammatory and | U.S. Pat. No. 5,608,035; U.S. Pat. No. |
| (linear) | autoimmune diseases | 5,786,331; U.S-Pat. No. 5,880,096; |
| | (“IL-1 antagonist” or “IL- | Yanofsky 91-al. (1996) PNAS |
| | 1 ra-mimetic”) | 93: 7381-6; Akeson et al. (1996), J. |
| | | Biol. Chem. 271: 30517-23; |
| | | Wiekzorek et al. (1997), Pol. J. |
| | | Pharmacol. 49: 107-17; Yanofsky |
| | | (1996), PNAs, 93: 7381-7386. |
| Facteur thyrnique | stimulation of | Inagaki-Ohara et al. (1996), Cellular |
| (linear) | lymphocytes (FTS- | Immunol. 171: 30-40; Yoshida (1984), |
| | mimetic) | J. Immunopharmacol, 6: 141-6. |
| CTLA4 MAb | CTLA4-mimetic | Fukumoto et al. (1998), Nature |
| (intrapeptide di-sulfide | | Biotech. 16: 267-70 |
| bonded) | | |
| TNF-a receptor | TNF-a antagonist | Takasaki et al. (1997), Nature Biotech. |
| (exo-cyclic) | | 15: 1266-70; WO 98/53842, published |
| | | Dec. 3, 1998. |
| TNF-a receptor | TNF-a antagonist | Chirinos-Rojas J. Imm., 5621-26. |
| (linear) | | |
| C3b | inhibition of complement | Sahu et al. (1996), Immunol. 157: 884-91; |
| (intrapeptide di-sulfide | activation; autoimmune | Morikis et al. (1998), Protein Sci. |
| bonded) | diseases (C3b antagonist) | 7: 619-27. |
| vinculin | cell adhesion processes, | Adey et al. (1997), Biochem. J. |
| (linear) | cell growth, differentiation | 324: 523-8 |
| | wound healing, tumor | |
| | metastasis (“vinculin | |
| | binding”) | |
| C4 binding protein (C413P) | anti-thrombotic | Linse et al. 272 Biol. Chem. 14658-65 |
| (linear) | | (1997) |
| urokinase receptor | processes associated with | Goodson et al. (1994), 91 PNAS 7 129-33; |
| (linear) | urokinase interaction with | International application WO |
| | its receptor (e.g. | 97/35969, published Oct. 2, 1997 |
| | angiogenesis, tumor cell | |
| | invasion and metastasis; | |
| | (URK antagonist) | |
| Mdm2, Hdm2 | Inhibition of inactivation | Picksley et al. (1994), Oncogene 9: |
| (linear) | of p53 mediated by Mdm2 | 2523-9; Bottger et al. (1997) J. Mol. |
| | or hdm2; anti-tumor | Biol. 269: 744-56; Bottger et al. |
| | (“Mdm/hdm antagonist”) | (1996), Oncogene 13: 2141-7 |
| p21^{WAF1} | anti-tumor by mimicking | Ball et al. (1997), Curr. Biol. 7: 71-80. |
| (linear) | the activity of p21^{WAF1} | |
| farnesyl transferase | anti-cancer by preventing | Gibbs et al. (1994), Cell 77: 175-178 |
| (linear) | activation of ras oncogene | |
| Ras effector domain | anti-cancer by inhibiting | Moodie et at. (1994), Trends Genel |
| (linear) | biological function of the | 10: 44-48 Rodriguez et al. (1994), |
| | ras oncogene | Nature 370: 527-532. |
| SH2/SH3 domains | anti-cancer by inhibiting | Pawson et al (1993), Curr. Biol. |
| (linear) | tumor growth with | 3: 434-432, Yu et al. (1994), Cell |
| | activated tyrosine kinases | 76: 933-945. |
| p16^{INK4} | anti-cancer by mimicking | Fahraeus et al. (1996), Curr. Biol. |
| (linear) | activity of p16; e.g., | 6: 84-91 |
| | inhibiting cyclin D-Cdk | |
| | complex (“p,16-mimetic”) | |
| Src, Lyn | inhibition of Mast cell | Stauffer et al. (1997), Biochem. 36: |
| (linear) | activation, IgE-related | 9388-94. |
| | conditions, type I | |
| | hypersensitivity (“Mast | |
| | cell antagonist”). | |
| Mast cell protease | treatment of inflammatory | International application WO |
| (linear) | disorders mediated by | 98/33812, published Aug. 6, 1998 |
| | release of tryptase-6 | |
| | (“Mast cell protease | |
| | inhibitors”) | |
| SH3 domains | treatment of SH3- | Rickles et al. (1994), EMBO |
| (linear) | mediated disease states | J. 13: 5598-5604; Sparks aLal. |
| | (“SH3 antagonist”) | (1994), J. Biol. Chem. 269: |
| | | 238536; Sparks et al. (1996), |
| | | PNAS 93: 1540-44. |
| HBV core antigen (HBcAg) | treatment of HBV viral | Dyson & Muray (1995), Proc. |
| (linear) | antigen (HBcAg) | NatI. Acad. Sci. 92: 2194-98. |
| | infections (“anti-HBV”) | |
| selectins | neutrophil adhesion | Martens et al. (1995), J. Biol. |
| (linear) | inflammatory diseases | Chem. 270: 21129-36; |
| | (“selectin antagonist”) | European pat. app. EP 0 714 |
| | | 912, published Jun. 5, 1996 |
| calmodulin | calmodulin | Pierce et al. (1995), Molec. |
| (linear, cyclized) | antagonist | Divemily 1: 25965; Dedman |
| | | et al. (1993), J. Biol. Chem. |
| | | 268: 23025-30; Adey & Kay |
| | | (1996), Gene 169: 133-34. |
| integrins | tumor-homing; treatment | International applications WO |
| (linear, cyclized) | for conditions related to | 95/14714, published Jun. 1, 1995; |
| | integrin-mediated cellular | WO 97/08203, published Mar. |
| | events, including platelet | 6, 1997; WO 98/10795, published |
| | aggregation, thrombosis, | Mar. 19, 1998; WO 99/24462, |
| | wound healing, | published May 20, 1999; Kraft et al. |
| | osteoporosis, tissue repair, | (1999), J. Biol. Chem. 274: 1979-85. |
| | angiogenesis (e.g., for | |
| | treatment of cancer) and | |
| | tumor invasion (“integrin- | |
| | binding”) | |
| fibronectin and | treatment of inflammatory | WO 98/09985, published Mar. 12, |
| extracellular matrix | and autoimmune | 1998. |
| components of T-cells and | conditions | |
| macrophages | | |
| (cyclic, linear) | | |
| somatostatin and cortistatin | treatment or prevention of | European patent application 0 911 |
| (linear) | hormone-producing | 393, published Apr. 28, 1999. |
| | tumors, acromegaly, | |
| | giantism, dementia, gastric | |
| | ulcer, tumor growth, | |
| | inhibition of hormone | |
| | secretion, modulation of | |
| | sleep or neural activity | |
| bacterial lipopoly-saccharide | antibiotic; septic shock; | U.S. Pat. No. 5,877,151, issued Mar. |
| (linear) | disorders modulatable by | 2, 1999. |
| | CAP37 | |
| parclaxin, mellitin | antipathogenic | WO 97/31019, published 28 Aug. |
| (linear or cyclic) | | 1997. |
| VIP | impotence, neuro- | WO 97/40070, published October 30, |
| (linear, cyclic) | degenerative disorders | 1997. |
| CTLs | cancer | EP 0 770 624, published May |
| (linear) | | 2, 1997. |
| THF-gamma2 | | Burnstein (1988), Biochem., 27: 4066-71 |
| (linear) | | |
| Amylin | | Cooper (1987), PNAS 84: 8628-32. |
| (linear) | | |
| Adreno-medullin | | Kitamura (1993), BBRC, 192: 553-60 |
| (linear) | | |
| VEGF | anti-angiogenic; cancer, | Fairbrother (1998), Biochem., |
| (cyclic, linear) | rheumatoid arthritis, | 37: 17754-64. |
| | diabetic retinopathy, | |
| | psoriasis (“VEGF | |
| | antagonist’”) | |
| MMP | inflammation and | Koivunen 17 Nature Biotech., 768-74 |
| (cyclic) | autoimmune disorders; | (1999). |
| | tumor growth (“MMP | |
| | inhibitor”) | |
| HGH fragment | | U.S. Pat. No. 5,869,452. |
| (linear) | | |
| Echistatin | inhibition of platelet | Gan (1988), J. Biol. 263: 19827-32. |
| | aggregation | |
| SLE autoantibody | SLE | WO 96/30057, published Oct. 3, 1996. |
| (linear) | | |
| GD1 alpha | suppression of tumor | Ishikawa et al., 1 FEBS Lett. 20-4 |
| | metastasis | (1998). |
| anti-phospholipid β-2 | endothelial cell activation, | Blank Mal. (1999), PNAS 96: 5164-8. |
| glycoprotein-1 (β2GPI) | anti-phospholipid | |
| antibodies | syndrome (APS), | |
| | thromboembolic | |
| | phenomena, | |
| | thrombocytopenia, and | |
| | recurrent fetal loss | |
| T-Cell Receptor β chain | diabetes | WO 96/101214, published Apr. 18, |
| (linear) | | 1996. |

There are two pivotal cytokines in the pathogenesis of rheumatoid arthritis, IL-1 and TNF-α. They act synergistically to induce each other, other cytokines, and COX-2. Research suggests that IL-1 is a primary mediator of bone and cartilage destruction in rheumatoid arthritis patients, whereas TNF-α appears to be the primary mediator of inflammation.

In a preferred embodiment of the invention, the polypeptide of interest binds to tumor necrosis factor alpha (TNFα), a pro-inflamatory cytokine. U.S. Pat. No. 6,277,969, issued Aug. 21, 2001; U.S. Pat. No. 6,090,382, issued Jul. 10, 2000. Anti-TNFα antibodies have shown great promise as therapeutics. For example, Infliximab, provided commercially as REMICADE® by Centocor, Inc. (Malvern, Pa.) has been used for the treatment of several chronic autoimmune diseases such as Crohn's disease and rheumatoid arthritis. Treacy, 19(4) Hum. Exp. Toxicol. 226-28 (2000); see also Chantry, 2(1) Curr. Opin. Anti-Inflammatory Immunomodulatory Invest. Drugs 31-34 (2000); Rankin et al., 34(4) Brit. J. Rheumatology 334-42 (1995). Preferably, any exposed amino acids of the TNFα-binding moiety of the polypeptide of interest are those with minimal antigenicity in humans, such as human or humanized amino acid sequences. These peptide identities may be generated by screening libraries, as described above, by grafting human amino acid sequences onto murine-derived paratopes (Siegel et al., 7(1) Cytokine 15-25 (1995); WO 92/11383, published Jul. 9, 1992) or monkey-derived paratopes (WO 93/02108, published Feb. 4, 1993), or by utilizing xenomice (WO 96/34096, published Oct. 31, 1996). Alternatively, murine-derived anti-TNFα antibodies have exhibited efficacy. Saravolatz et al., 169(1) J. Infect. Dis. 214-17 (1994).

Alternatively, instead of being derived from an antibody, the TNFα binding moiety of the polypeptide of interest may be derived from the TNFα receptor. For example, Etanercept is a recombinant, soluble TNFα receptor molecule that is administered subcutaneously and binds to TNFα in the patient's serum, rendering it biologically inactive. Etanercept is a dimeric fusion protein consisting of the extracellular ligand-binding portion of the human 75 kilodalton (p75) tumor necrosis factor receptor (TNFR) linked to the Fc portion of human IgG1. The Fc component of etanercept contains the C_{H}2 domain, the C_{H}3 domain and hinge region, but not the C_{H}1 domain of IgG1. Etanercept is produced by recombinant DNA technology in a Chinese hamster ovary (CHO) mammalian cell expression system. It consists of 934 amino acids and has an apparent molecular weight of approximately 150 kilodaltons. Etanercept may be obtained as ENBREL™, manufactured by Immunex Corp. (Seattle, Wash.). Etanercept may be efficacious in rheumatoid arthritis. Hughes et al., 15(6) Biodrugs 379-93 (2001).

Another form of human TNF receptor exists as well, identified as p55. Kalinkovich et al., J. Inferon & Cytokine Res. 15749-57 (1995). This receptor has also been explored for use in therapy. See, e.g., Qian et al. 118 Arch. Ophthalmol. 1666-71 (2000). A previous formulation of the soluble p55 TNF receptor had been coupled to polyethylene glycol [r-metHuTNFbp PEGylated dimer (TNFbp)], and demonstrated clinical efficacy but was not suitable for a chronic indication due to the development antibodies upon multiple dosing, which resulted in increased clearance of the drug. A second generation molecule was designed to remove the antigenic epitopes of TNFbp, and may be useful in treating patients with rheumatoid arthritis. Davis et al., Presented at the Ann. European Cong. Rheumatology, Nice, France (Jun. 21-24, 2000).

IL-1 receptor antagonist (IL-1 Ra) is a naturally occurring cytokine antagonist that demonstrates anti-inflammatory properties by balancing the destructive effects of IL-1α and IL-1β in rheumatoid arthritis but does not induce any intracellular response. Hence, in a preferred embodiment of the invention, the polypeptide of interest comprises IL-1Ra, or any structural or functional analog thereof. Two structural variants of IL-1Ra exist: a 17-kDa form that is secreted from monocytes, macrophages, neutrophils, and other cells (sIL-1Ra) and an 18-kDa form that remains in the cytoplasm of keratinocytes and other epithelial cells, monocytes, and fibroblasts (icIL-1Ra). An additional 16-kDa intracellular isoform of IL-1Ra exists in neutrophils, monocytes, and hepatic cells. Both of the major isoforms of IL-1Ra are transcribed from the same gene through the use of alternative first exons. The production of IL-1Ra is stimulated by many substances including adherent IgG, other cytokines, and bacterial or viral components. The tissue distribution of IL-1Ra in mice indicates that sIL-1Ra is found predominantly in peripheral blood cells, lungs, spleen, and liver, while icIL-1Ra is found in large amounts in skin. Studies in transgenic and knockout mice indicate that IL-1Ra is important in host defense against endotoxin-induced injury. IL-1Ra is produced by hepatic cells with the characteristics of an acute phase protein. Endogenous IL-1Ra is produced in human autoimmune and chronic inflammatory diseases. The use of neutralizing anti-IL-1Ra antibodies has demonstrated that endogenous IL-1Ra is an important natural anti-inflammatory protein in arthritis, colitis, and granulomatous pulmonary disease. Patients with rheumatoid arthritis treated with IL-1Ra for six months exhibited improvements in clinical parameters and in radiographic evidence of joint damage. Arend et al., 16 Ann. Rev. Immunol. 27-55 (1998).

Yet another example of an IL-1Ra that may be expressed by the cell clone of the present invention is a recombinant human version called interleukin-117.3 Kd met-IL 1ra, or Anakinra, produced by Amgen, (San Francisco, Calif.) under the name KINERET™. Anakinra has also shown promise in clinical studies involving patients with rheumatoid arthritis (Presented at the 65th Ann. Sci. Meeting of Am. College Rheumatology. Nov. 12, 2001).

In another embodiment of the invention, the polypeptide of interest expressed by the cell clone of the present invention is interleukin 12 (IL-12) or an antagnoist thereof. IL-12 is a heterodimeric cytokine consisting of glycosylated polypeptide chains of 35 and 40 kD which are disulfide bonded. The cytokine is synthesized and secreted by antigen presenting cells, including dendritic cells, monocytes, macrophages, B cells, Langerhans cells and keratinocytes, as well as natural killer (NK) cells. IL-12 mediates a variety of biological processes and has been referred to as NK cell stimulatory factor (NKSF), T-cell stimulating factor, cytotoxic T-lymphocyte maturation factor and EBV-transformed B-cell line factor. Curfs et al., 10 Clin. Micro. Rev. 742-80 (1997). Interleukin-12 can bind to the IL-12 receptor expressed on the plasma membrane of cells (e.g., T cells, NK cell), thereby altering (e.g., initiating, preventing) biological processes. For example, the binding of IL-12 to the IL-12 receptor can stimulate the proliferation of pre-activated T cells and NK cells, enhance the cytolytic activity of cytotoxic T cells (CTL), NK cells and LAK (lymphokine activated killer) cells, induce production of gamma interferon (IFN GAMMA) by T cells and NK cells and induce differentiation of naive Th0 cells into Th1 cells that produce IFN GAMMA and IL-2. Trinchieri, 13 Ann. Rev. Immunology 251-76 (1995). In particular, IL-12 is vital for the generation of cytolytic cells (e.g., NK, CTL) and for mounting a cellular immune response (e.g., a Th1 cell mediated immune response). Thus, IL-12 is critically important in the generation and regulation of both protective immunity (e.g., eradication of infections) and pathological immune responses (e.g., autoimmunity). Hendrzak et al., 72 LAB. Investigation 619-37 (1995). Accordingly, an immune response (e.g., protective or pathogenic) can be enhanced, suppressed or prevented by manipulation of the biological activity of IL-12 in vivo, for example, by means of an antibody.

In another embodiment of the present invention, the polypeptide of interest comprises or targets an integrin. Integrins have been implicated in the angiogenic process, by which tumor cells form new blood vessels that provide tumors with nutrients and oxygen, carry away waste products, and to act as conduits for the metastasis of tumor cells to distant sites, Gastl et al., 54 Oncol. 177-84 (1997). Integrins are heterodimeric transmembrane proteins that play critical roles in cell adhesion to the extracellular matrix (ECM) which, in turn, mediates cell survival, proliferation and migration through intracellular signaling. During angiogenesis, a number of integrins that are expressed on the surface of activated endothelial cells regulate critical adhesive interactions with a variety of ECM proteins to regulate distinct biological events such as cell migration, proliferation and differentiation. Specifically, the closely related but distinct integrins aVb3 and aVb5 have been shown to mediate independent pathways in the angiogenic process. An antibody generated against αVβ3 blocked basic fibroblast growth factor (bFGF) induced angiogenesis, whereas an antibody specific to αVβ5 inhibited vascular endothelial growth factor-induced (VEGF-induced) angiogenesis. Eliceiri et al., 103 J. Clin. Invest. 1227-30 (1999); Friedlander et al., 270 Science 1500-02 (1995).

In another preferred embodiment of the invention, the polypeptide of interest comprises at least one glycoprotein IIb/IIIa receptor antagonist. More specifically, the final obligatory step in platelet aggregation is the binding of fibrinogen to an activated membrane-bound glycoprotein complex, GP IIb/IIIa. Platelet activators such as thrombin, collagen, epinephrine or ADP, are generated as an outgrowth of tissue damage. During activation, GP IIb/IIIa undergoes changes in conformation that results in exposure of occult binding sites for fibrinogen. There are six putative recognition sites within fibrinogen for GP IIb/IIIa and thus fibrinogen can potentially act as a hexavalent ligand to crossing GP IIb/IIIa molecules on adjacent platelets. A deficiency in either fibrinogen or GP IIb/IIIa a prevents normal platelet aggregation regardless of the agonist used to activate the platelets. Since the binding of fibrinogen to its platelet receptor is an obligatory component of normal aggregation, GP IIb/IIIa is an attractive target for an antithrombotic agent.

Results from clinical trials of GP IIb/IIIa inhibitors support this hypothesis. A Fab fragment of the monoclonal antibody 7E3, which blocks the GP IIb/IIIa receptor, has been shown to be an effective therapy for the high risk angioplasty population. It is used as an adjunct to percutaneous transluminal coronary angioplasty or atherectomy for the prevention of acute cardiac ischemic complications in patients at high risk for abrupt closure of the treated coronary vessel. Although 7E3 blocks both the IIb/IIIa receptor and the (α_{V}β₃ receptor, its ability to inhibit platelet aggregation has been attributed to its function as a IIb/IIIa receptor binding inhibitor. The IIb/IIIa receptor antagonist may be, but is not limited to, an antibody, a fragment of an antibody, a peptide, or an organic molecule. For example, the target-binding moiety may be derived from 7E3, an antibody with glycoprotein IIb/IIIa receptor antagonist activity. 7E3 is the parent antibody of c7E3, a Fab fragment known as abciximab, known commercially as REOPRO® produced by Centocor, Inc. (Malvern, Pa.).

Abciximab binds and inhibits the adhesive receptors GPIIb/IIIa and α_{V}β₃, leading to inhibition of platelet aggregation and thrombin generation, and the subsequent prevention of thrombus formation. U.S. Pat. Nos. 5,976,532, 5,877,006, 5,770,198; Coller, 78 Throm Haemost. 730-35 (1997); Jordan et al., in Adhesion Receptors as Therapeutic Targets 281-305 (Horton, ed. CRC Press, New York, 1996); Jordan et al., in New Therapeutic Agents in Thrombosis & Thrombolysis (Sasahara & Loscalzo, eds. Marcel Kekker, Inc. New York, 1997).

Additionally, the glycoprotein IIb/IIIa receptor antagonist expressed by the cell clone of the present invention may comprise a thrombolytic. For example, the thrombolytic may be tPA, or a functional variation thereof. RETAVASE®, produced by Centocor, Inc. (Malvern, Pa.), is a variant tPA with a prolonged half-life. In mice, the combination of Retavase and the IIb/IIIa receptor antagonist c7E3 Fab markedly augmented the dissolution of pulmonary embolism. See Provisional Patent Application Ser. No. 60/304,409.

Alternatively, the method of the present invention can be used to identify cell clones secreting non-peptide molecules. For example, natural signaling molecules are endogenous compounds which chemically effect receptors. Many pharmacologically active drugs act on the cellular receptor level by either mimicking the action of a natural signal molecule (agonist) or by blocking the action of the natural signal molecule (antagonist). As a non-limiting example, tirofiban hydrochloride is a non-peptide antagonist of the platelet glycoprotein IIb/IIIa receptor that inhibits platelet aggregation. See U.S. Pat. No. 6,117,842, issued Sep. 12, 2000. Tirofiban is commercially available as AGGRASTAT® from Merck & Co., Inc., (Whitehouse Station, N.J.), manufactured by Baxter Healthcare Corp. (Deerfield, Ill.) and Ben Venue Labs. (Bedford, Ohio). The structure of Tirofiban is illustrated below where X is or contains a functional group capable of forming the ΨAb structure. The position of X is selected at any of those aromatic sites on the molecule for which substitution will retain some activity of the parent structure, and is not limited to that position depicted in the drawing.

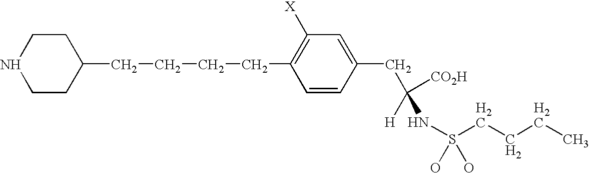

The polypeptide of interest expressed by the cell clone of the present invention also include receptors or fragments thereof, and activated receptors, i.e., recombinant peptides that mimic ligands associated with their corresponding receptors, or fragments thereof. These complexes may mimic activated receptors and thus affect a particular biological activity. An example of activated-receptor moieties concerns the peptido mimetics of the erythropoietin (Epo) receptor. By way of background, the binding of Epo to the Epo receptor (EpoR) is crucial for production of mature red blood cells. The Epo-bound, activated EpoR is a dimer. See, e.g., Constantinescu et al., 98 PNAS 4379-84 (2001). In its natural state, the first EpoR in the dimer binds Epo with a high affinity whereas the second EpoR molecule binds to the complex with a low affinity. Bivalent anti-EpoR antibodies have been reported to activate EopR, probably by dimerization of the EpoR. Additionally, small synthetic peptides, that do not have any sequence homology with the Epo molecule, are also able to mimic the biologic effects of Epo but with a lower affinity. Their mechanism of action is probably also based on the capacity to produce dimerization of the EpoR. Hence, an embodiment of the present invention provides for a method of identifying and characterizing cell clones expressing an activated EpoR mimetic.

In another preferred embodiment, the method of the present invention may be used to identify cell clone that secrets antimicrobial agents or portions thereof, which include antibacterial agents, antivirals agents, antifungal agents, antimycobacterial agents, and antiparasitic agents. Antibacterials include, but are not limited to, Beta-lactams (such as Penicillins and Cephalosporins), Aminoglycosides (such as Gentamicin), Macrolides (such as Erythromycin), Fluoroquinolones, Metronidazole, Sulfonamides, Tetracyclines, Trimethroprim, and Vancomycin. Antifungal agents include, but are not limited to Amphotericin, Fluconazole, Flucytosine, Itraconazole, and Ketoconazole. Antiparasitic agents include, but are not limited to, Ivermectin, Mebendazole, Mefloquine, Pentamidine, Praziquantel, Pyrimethamine, and Quinine. Antiviral agents include, but are not limited to, Acyclovir, Amantadine, Didanosine, Famciclovir, Foscarnet, Ganciclovir, Rimatandine, Stavudine, Zalcitabine, and Zidovudine. Antimycobacterial agents include, but are not limited to, Isoniazid, Rifampin, Streptomycin, Dapsone. Sanford et al., Guide to Antimicrobial Therapy (25th ed., Antimicrobial Therapy, Inc., Dallas, Tex. 1995).

The method of the present invention may also be used to identify and/or characterize cell clones expressing a particular antigen. Antigens, in a broad sense, may include any molecule to which an antibody, or functional fragment thereof, binds. Such antigens may be pathogen derived, and be associated with either MHC class I or MHC class II reactions. These antigens may be proteinaceous or include carbohydrates, such as polysaccharides, glycoproteins, or lipids. Carbohydrate and lipid antigens are present on cell surfaces of all types of cells, including normal human blood cells and foreign, bacterial cell walls or viral membranes. Nucleic acids may also be antigenic when associated with proteins, and are hence included within the scope of antigens encompassed in the present invention. See Sears, Immunology (W. H. Freeman & Co. and Sumanas, Inc., 1997), available on-line at http://www.whfreeman.com/immunology. For example, antigens may be derived from a pathogen, such as a virus, bacterium, mycoplasm, fungus, parasite, or from another foreign substance, such as a toxin. Such bacterial antigens may include or be derived from Bacillus anthracis, Bacillus tetani, Bordetella pertusis; Brucella spp., Corynebacterium diphtheriae, Clostridium botulinum, Clostridium perfringens, Coxiella burnetii, Francisella tularensis, Mycobacterium leprae, Mycobacterium tuberculosis, Salmonella typhimurium, Streptocccus pneumoniae, Escherichia coli, Haemophilus influenzae, Shigella spp., Staphylococcus aureus, Neisseria gonorrhoeae, Neisseria meningiditis, Treponema pallidum, Yersinia pestis, Vibrio cholerae. Often, the oligosaccharide structures of the outer cell walls of these microbes afford superior protective immunity, but must be conjugated to an appropriate carrier for that effect.

Viruses and viral antigens that are within the scope of the current invention include, but are not limited to, HBeAg, Hepatitis B Core, Hepatitis B Surface Antigen, Cytomegalovirus B, HIV-1 gag, HIV-1 nef, HIV-1 env, HIV-1 gp41-1, HIV-1 p24, HIV-1 MN gp120, HIV-2 env, HIV-2 gp 36, HCV Core, HCV NS4, HCV NS3, HCV p22 nucleocapsid, HPV L1 capsid, HSV-1 gD, HSV-1 gG, HSV-2 gG, HSV-II, Influenza A (H1N1), Influenza A (H3N2), Influenza B, Parainfluenza Virus Type 1, Epstein Barr virus capsid antigen, Epstein Barr virus, Poxyiridae Variola major, Poxyiridae Variola minor, Rotavirus, Rubella virus, Respiratory Syncytial Virus, Surface Antigens of the Syphilis spirochete, Mumps Virus Antigen, Varicella zoster Virus Antigen and Filoviridae.

Other parasitic pathogens such as Chlamydia trachomatis, Plasmodium falciparum, and Toxoplasma gondii may also be included in the scope of the present invention. Numerous bacterial and viral, and other microbe-generated antigens are available from commercial suppliers such as Research Diagnostics, Inc. (Flanders, N.J.).

Toxins, toxoids, or antigenic portions of either, within the scope of the present invention include those produced by bacteria, such as diphteria toxin, tetanus toxin, botulin toxin and enterotoxin B; those produced by plants, such as Ricin toxin from the castor bean Ricinus cummunis. Mycotoxins, produced by fungi, that may serve in the present invention include diacetoxyscirpenol (DAS), Nivalenol, 4-Deoxynivalenol (DON), and T-2 Toxin. Other toxins and toxoids produced by or derived from other organisms may also be included in the scope of the present invention.

### Vectors

In a preferred embodiment, the cell clone of the present invention expresses at least one polypeptide of interest in detectable amount. A variety of mammalian expression vectors may be used to express the polypeptide of interest in the cell clone of the present invention. Expression vectors will preferably but optionally include at least one selectable marker. Such markers include, e.g., but not limited to, methotrexate (MTX), dihydrofolate reductase (DHFR, U.S. Pat. Nos. 4,399,216; 4,634,665; 4,656,134; 4,956,288; 5,149,636; 5,179,017, ampicillin, neomycin (G418), mycophenolic acid, or glutamine synthetase (GS, U.S. Pat. Nos. 5,122,464; 5,770,359; 5,827,739) resistance for eukaryotic cell culture, and tetracycline or ampicillin resistance genes for culturing in E. coli and other bacteria or prokaryotics (the above patents are entirely incorporated hereby by reference).

Suitable vectors are readily apparent to the skilled artisan. For example, commercially available mammalian expression vectors that may be suitable for the present invention include, but are not limited to, pMAMneo (Clontech, Palo Alto, Calif.), pcDNA3 (Invitrogen, Carlsbad, Calif.), pMClneo (Stratagene, La Jolla, Calif.), pXTI (Stratagene, La Jolla, Calif.), pSG5 (Stratagene, La Jolla, Calif.), EBO-pSV2-neo (ATCC, Manassas, Va., ATCC No. 37593), pBPV-1 (8-2) (ATCC No. 37110), pdBPV-MMTneo (342-12) (ATCC No. 37224), pRSVgpt (ATCC No.] 37199), pRSVneo (ATCC No. 37198), pSV2-dhfr (ATCC No. 37146), pUCTag (ATCC No. 37460), and 17D35 (ATCC No. 37565).

The nucleic acid encoding at least one polypeptide of interest may be introduced by one of several methods well known in the art, including but not limited to, transfection, including but not limited to, calcium phosphate transfection, DEAE-dextran mediated transfection and cationic lipid-mediated transfection, electroporation, sonication, transduction, transformation, and viral infection. Such methods are described in the art, see, e.g., Samsrook et al., Molecular Cloning: a Lab Manual, 3rd edition, Cold Spring Harbor, N.Y. (2001); Ausubel et al., Current Protocols in Molecular Biology, John Wiley & Sons, Inc., NY, N.Y. (1987-2007).

### Host Cell Lines

The host cells in the present invention can be at least one selected from prokaryotic or eukaryotic cells, or fusion cells thereof, e.g., but not limited to, bacterial cells, blue-green algae cells, yeast cells, silk worm cells, plant cells, insect cells, amphibian cells, fish cells, avian cells, mammalian cells, or any derivative, immortalized or transformed cell thereof. Preferably, the cells are eukaryotic cells. More preferably, the cells are mammalian cells.

In a preferred embodiment, suitable cell lines that can be used according to the present invention include any transformed or immortalized mammalian cell line. The host cell can optionally be at least one selected from myeloma cells, such as but not limited to Sp2/0, NSO, NS1, CHO, BHK, Ag653, P3X63Ag8.653 (ATCC Accession Number CRL-1580) and SP2/0-Ag14 (ATCC Accession Number CRL-1851), COS-1 (e.g., ATCC CRL-1650), COS-7 (e.g., ATCC CRL-1651), HEK293, BHK21 (e.g., ATCC CAL-10), CHO (e.g., ATCC CRL-1610, CHO DXB-11, CHO DG44), BSC-1 (e.g., ATCC CAL-26), HepG2, 293, HeLa, NIH 3T3, CDS-1, CDS-7, NIH 273, or lymphoma cells, or any derivative, immortalized or transformed cell thereof. A preferred cell line is C463A, which is derived from Sp2/0 and can be used as a transfection host. See U.S. application 60/339,428, WO2003051720 and WO993052964, herein entirely incorporated by reference.

As used herein, the term “colony” or “colonies” may be defined by the number of cells or total diameter, which is determined by the researcher. Typically, a colony has at least 40 or 50 cells, although sometimes as few as 30 cells or less. The incubation period required for a given cell type to reach the critical size or number of cells to be called a colony varies between cell types, but typically requires an incubation period of between 7-14 days, with longer periods needed if the cell growth is slow. If diameter is used as the defining criterion, a colony is typically defined as being 10-50 microns, such as 10-20, 20-30, 30-40, 40-50 microns or any range or value therein.

### Media

Appropriate culture media and conditions for the above-described host cells are well known in the art. Numerous types of growth media are commercially available, such as but not limited to Iscove's Modified Medium, Dulbecco's Modified Eagel Medium, RPMI, Ham's F10, Ham's F12, Minimum Essetial Medium and alpha medium etc. In addition to growth media, cells cultured in vitro require many growth factors to either promote growth or maintain viability. The growth factors may be supplied by for example, 5-10% fetal bovine serum (FBS) to promote cell growth and protein production. However, cell growth media include serum-free (containing 0-0.5% serum) or serum-reduced (containing 0.5-5.0% serum) media.

To support the growth of mammalian cells, a variety of components, e.g. but not limited to, glutamine, glucose, vitamins, amino acids and growth factors, may be included in the culture media. Trace elements such as zinc, iron, selenium, copper, molybdenum, and manganese etc. are important for cloning and continuous passage of mammalian cells in stringent conditions of serum-free media. Alternatively, cell growth media include deficient media, where one or more nutrients are deleted. Growth media also include specialty media which are designed to promote growth of specific cell types.

Growth media may include additional antibiotics, attachment and matrix factors which are usually added to facilitate attachment and spreading of many types of anchorage dependent cells. Buffers may also be added to growth media in order to maintain pH levels. Such buffers may include but are not limited to MOPS, HEPES, sodium phosphate, potassium phosphate, Tris or other known buffers.

In addition, chemically defined media (CDM) can be used in the present invention.

CDM provide certain compounds, amino acids, lipids, carbohydrates, trace elements and/or vitamins and exclude the use of non-defined animal derived raw materials, e.g. but not limited to, primatone, albumin and Excyte™, as well as other similar materials derived from serum or other animal derived proteins or products. Such media allow the growth of cells to provide commercially useful amounts of the desired proteins expressed in such cell cultures. Some of the advantages of CDM include but not limited to better protein producing, commercially suitable, cost-effective, and/or pose reduced regulatory concerns for proteins produced in cell lines grown therein. For detailed compositions and formulations of CDM, see e.g. but not limited to WO2002066603, herein entirely incorporated by reference.

As used herein the term “semi-solid medium” refers to a cell growth medium that does not provide a solid substrate to which cells can attach, and that is sufficiently viscous such that cells added to the semi-solid medium are suspended therein, and are thereby prevented from sinking through the semi-solid medium and contacting, and attaching to, the inner surface of the container within which the semi-solid medium is dispensed. Because a semi-solid medium holds the cells in situ, it permits continuous observation of a single cell or individual colony. Such semi-solid media further comprise fluorescent protein A or G to enchance detection and recover of positive clones.

Semi-solid media useful in the practice of the present invention typically include a gelatinization agent dissolved in an aqueous medium in an amount of from 0.1% to 5.0% (w/v), such as 0.1-0.5%, 0.5-1.0%, 1.0-1.5%, 1.5-2.0%, 2.0-2.5%, 2.5-3.0%, 3.0-3.5%, 3.5-4.0%, 4.0-4.5%, 4.5-5.0% or any range or value therein. Preferred semi-solid media are those capable of sustaining growth of cells. Non-limiting examples of gelatinization agents include agar, agarose, methylcellulose, or any other polymer suitable for the purpose of the present invention.

One category of the semi-solid media forms a liquid at temperatures above room temperature or above the temperature required to incubate the cells, and forms a semi-solid or gel when at room temperature or the temperature at which the cells are incubated. For example, agar is a class of polysaccharide complex generally defined as a dried mucilaginous substance extracted from the agarocytes of algae of the Rhodophyceae. Agar-producing genera include but not limited to, Gelidium, Gracilaria, Acanthopeltis, Ceramim, Pterocladia etc. Agar melts at about 100° C. and solidifies into a gel at about 40° C. It is not digested by most bacteria. Agarose is a modified agar, whereby sugars, methyl groups, and other chemical groups are chemically bonded to agar in order to enhance desired physical properties, such as low gelling temperature.

Additional gelatinization agents include, but are not limited to a wide variety of polymers, including proteins and their derivatives, may be used as semi-solid matrices in the present invention. Matrigel®, collagen or gelatin, or other similar materials may also be used as the semi-solid matrix.

Methylcellulose (cellulose methyl ether) belongs to a group of compounds known as cellulose ethers. The cellulose ethers are manufactured by a reaction of purified cellulose with alkylating reagents (methyl chloride) in presence of a base, typically sodium hydroxide and an inert diluent. The addition of the base in combination with water activates the cellulose matrix by disrupting the crystalline structure and increasing the access for the alkylating agent and promotes the etherification reaction. This activated matrix is called alkali cellulose. Methylcellulose is prepared from wood pulp or chemical cotton by treatment with alkali and methylation of the alkali cellulose with methyl chloride that adds methyl ether groups. The reaction can be characterized as:

R_{cell}OH:NaOH+CH₃Cl→R_{cell}OCH₃+NaCl

One significant property of methylcellulose is its reversible thermal gelation: it is soluble in cold water but insoluble in hot water. An aqueous solution is best prepared by dispersing the granules in hot (but not boiling) water with stirring and chilling to +5° C. Presence of inorganic salts increases the viscosity. At room temperature, methylcellulose solution is stable and stays in semi-solid gel form. It supports mammalian cell growth when mixed with the proper growth medium. The viscosity of methylcellulose prevents aggregation of the cells. In one embodiment, the final concentration of methylcellulose in the semi-solid capture medium is 1%. In another embodiment, the final concentration is around 0.7%. Less methylcellulose in the medium allows better diffusion of the capture molecule and accordingly increases the detection sensitivity.

Alternatively, premixed methylcellulose based semi-solid media are commercially available, such as but not limited to, ClonaCell™-TCS and MethCult™ media (StemCell Technologies), Stemline™ methylcellulose media (Sigma-Aldrich, St. Louis, Mo.).

Addition of methylcellulose is traditionally used when culturing erythroid progenitor cells. The application of methylcellulose for screening and selection of antibiotic resistant clones has been described and commercially available, e.g. see Technical Manual ClonalCell™-TCS, Transfected Cell Selection Kit, Stemcell Technologies.

### Capture Molecule

As used herein the term “the capture molecule”, which can be optionally used to label the polypeptide of interest to provide for detection using fluorescent Protein A or Protein G flourescence, refers to a molecule that can bind or react with the polypeptide of interest and form a halo-like precipitate visible under a microscope. Potential capture molecule can be but are not limited to, receptor or ligand of the polypeptide of interest, antibody or antigen against the polypeptide of interest etc. Accordingly, as used herein the term “the capture medium” refers to the semi-solid cell growth medium with at least one capture molecule incorporated and which further comprises fluorescent protein A or G. to enhance detection.

The capture molecule can be directly added to the semi-solid medium, either by mixing it with the medium before pouring the plates, or by overlaying the pored plates with a layer of medium containing the capture molecule. The capture molecule can be further radio-labeled, fluorescent-labeled or labeled by any other methods known in the art to facilitate the detection of precipitate. For example, a capture antibody is fluorescent-labeled and added to the semi-solid medium. Upon binding to the polypeptide of interest (i.e., the antigen), the antigen-antibody complex can be easily observed under fluorescent microscope and the cell clone expressing the polypeptide of interest can be identified.

In one embodiment, the capture molecule is an antibody against the polypeptide of interest. The final concentration of the capture antibody used can be 0.0225-0.225 mg/ml, such as 0.0225-0.045, 0.045-0.0675, 0.0675-0.09, 0.09-0.1125, 0.1125-0.135, 0.135-0.1575, 0.1575-0.18, 0.18-0.2025, 0.2025-0.225 mg/ml, or any range or value therein. In a preferred embodiment, the final concentration of the capture antibody is 0.1125 mg/ml. In general, lower concentration of the capture molecule increases the detection sensitivity by selecting cell clones expressing the polypeptide of interest at higher levels.

In one variation of the aforedescribed methods, this strategy is used to screen a nucleic acid library, such as a cDNA library, that encodes a population of candidate protein molecules that are being screened for their ability to bind or to react with the capture molecule and form precipitate. The cDNA library is introduced into cells by means well known in the art, such as by transfection or transduction. The cells are cultured in a semi-solid medium, preferably a methylcellulose based medium, in which a capture molecule is added. The colonies around which a precipitated halo is observed can be isolated and further studied. The foreign DNA can be retrieved from such colonies to identify and isolate the capture binding/interacting molecule that was responsible for the formation of the precipitate halo.

### Isolating Polypeptide of Interest

In one embodiment, after the cell clone being identified, it is harvested and expanded in culture and the polypeptide of interest is isolated therefrom using techniques well established in the art. The polypeptide of interest preferably is recovered from the culture medium as a secreted polypeptide. As a first step, the culture medium is centrifuged to remove particulate cell debris. The polypeptide thereafter is purified from contaminant soluble proteins and polypeptides, with the following procedures being exemplary of suitable purification procedures: by fractionation on immunoaffinity or ion-exchange columns; ethanol precipitation; reverse phase HPLC; chromatography on silica or on a cation-exchange resin such as DEAE; chromatofoucsing; SDS-PAGE; ammonium sulfate precipitation; gel filtration etc. A protease inhibitor such as phenyl methyl sulfonyl fluoride (PMSF) also may be useful to inhibit proteolytic degradation during purification. Additionally, the polypeptide of interest can be fused in frame to a marker sequence, such as but not limited to a hexahistidine (HA) tag, which allows for purification of the polypeptide of interest.

The methods of the present invention are also useful in identifying cell clones expressing G-protein coupled receptors (GPCRs) and other transmembrane proteins. These proteins may be purified as part of a membrane fraction or purified from the membranes by methods known in the art.

### Advantage

In the present invention, cells producing the polypeptide of interest can be identified by reference to the formation of relative fluorescence of the amount of fluorescent Protein A or G bound to the polypeptide of interest, or optionally using a polypeptide capture molecule that bind the polypeptide and also binds to fluorescent Protein A or Protein G. It will be clear to the skilled artisan that one of the benefits of the present invention is that it eliminates intermediate steps normally required in conventional screening methods, such as ELISA. In addition, high level producers can be identified by reference to the flourescence. Therefore, the present invention provides a simple yet powerful qualitative and/or qualitative screening method in contrast to conventional methods, such as ELISA, which are largely quantitative. Accordingly, the method of the present invention can be used as the primary screening method to examine large number of cells and is less labor-intensive and less time-consuming.

It will also be clear to the skilled artisan that this method can be used in robotic screening and in protocols for high throughput selection of cells producing high levels of a product of interest.

A preferred embodiment of the present invention is described by reference to the following examples, which are provided by way of illustration and are not intended as limiting. In this embodiment exemplified below, selection can be visually monitored by the immunoprecipitate (halo) formed between the chimeric anti-TNF antibody cA2 and the capture antibody rabbit anti-human IgG (H&L), while the production level of cA2 correlates with the size of the halo.

### Example 1

### Preparation of Methylcellulose Based Semi-Solid Capture Medium with Capture Antibody

Pre-made semi-solid matrix (4000 cps) containing methylcellulose in growth medium such as IMDM, EMDM, CD CHO, CD Hybridoma are commercially available. For example, Methocult from StemCell Technologies was used in the following experiments.

The semi-solid capture medium was prepared by adding 1 ml capture antibody (2 mg/ml) to 13 ml methylcellulose medium. Cell suspension was added to the mixture along with FBS, L-glutamine and additional growth medium to make 20 ml of final volume. In this example, the final concentration of the components are 1% methylcellulose, 30% FBS and 2 mM L-glutamine. It is readily understood that other concentrations suitable for the specific cell line are within the scope of this invention.

This working mixture was placed in a proper container (such as a 50 ml conical centrifuge tube) and mixed or vortexed vigorously for 30 seconds. After mixing, the tubes sat at room temperature for 5-10 minutes to allow air bubbles to disappear. The 20 ml of cells in the capture medium was evenly dispensed into a 6-well plate. The plate was incubated in a 37° C. CO₂ incubator without disturbance for 7 to 10 days. The plates were then removed for examination.

The sensitivity of this assay can be optimized by changing the concentration of capture antibody and the amount of methylcellulose used to make the semi-solid capture medium. Combination of lower capture antibody concentration and less methylcellulose routinely result in better detection sensitivity.

### Example 2

### Identification of Antibody Producing Clones Using Fluorescent Protein A/G Based Secreted Antibody Detection Assay

Fluorescent protein A/G based secreted protein detection assay was first exemplified by a stable CHO cell line expressing a recombinant antibody (SM1.141.224). These cells were mixed in the 1:1 ratio with non-expressing CHO host cells in a custom Methocult formulation from Stem Cell Technologies (Cat. # M03999) containing 2.5% methylcellulose in Dulbecco's Modified Eagle's Medium (DMEM). CHO host cells served as internal negative control. Methocult was supplemented with additional reagents as indicated below in Table 5.

TABLE 5
Solution for plating

| Component | Amount |
|---|---|
| Methocult, Stem Cell Technologies Cat. # M03999 | 40% v/v |
| Advanced DMEM/F12, Invitrogen Cat. #12634-010 | |
| Dialyzed FBS, Hyclone Cat. #SH30079.03 | 30% v/v |
| Alexa Fluor 488 protein A/G, Invitrogen Cat. #P11047 | See |
| (protein A)/P11065(protein G) 1 mg/mL reconstituted in | description |
| PBS | |
| Glutamine Synthetase (GS) supplement, 50X stock, | 2% v/v |
| JRH Cat. # 58672-100M | |
| Number of cells (1:1 ratio of C1013A and SM1.141.224) | 72 cells/mL |
| Q.S. with Advanced DMEM/F12, Invitrogen Cat. | 100% v/v |
| #12634-010 to | |

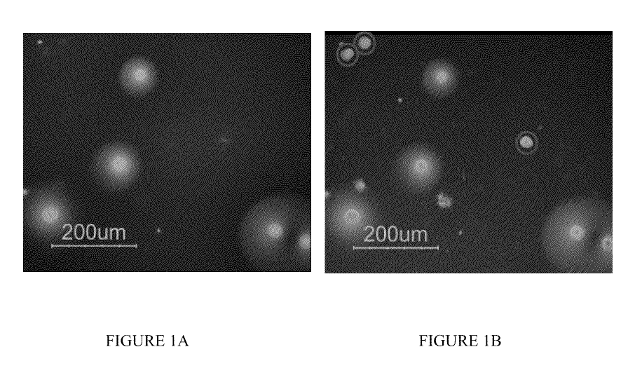

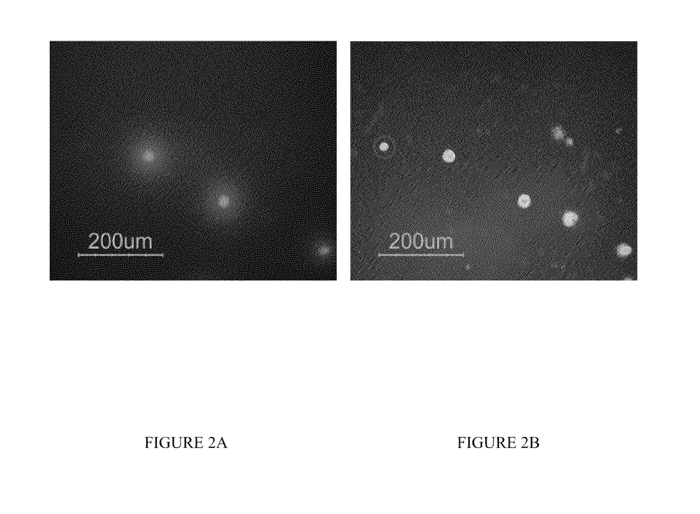

Multiple concentrations of Alexa Fluor 488 protein A/G were used to determine optimal concentration. Alexa Fluor 488 protein A was tested at 3, 5, 7, 9, 11, and 13 ug/mL, while Alexa Fluor 488 protein G was tested at 6, 8, 10, 12, 14, and 16 ug/mL. After addition of all reagents, the solution was mixed vigorously for 30 seconds. After mixing, 2 mL of solution was added to each well of a six-well plate. The plates were incubated undisturbed for 7 to 12 days at 37° C. with 5% CO₂. After eight days of incubation, a fluorescent microscope was used to visualize fluorescence. Fluorescent and white-light images were taken with a digital camera connected to the microscope to document fluorescent and non-fluorescent colonies. Representative fluorescent and white-light images are shown in FIG. 1 and FIG. 2. Specific bright green fluorescence on or around the cell colonies was clearly visible in the presence of Alexa Fluor 488 protein A/G. Some of the cells did not exhibit any fluorescence. Non-fluorescent colonies are presumed to be originated from parental CHO cells, which do not secrete any antibody. To verify this, six fluorescent and six non-fluorescent colonies were picked from the protein A experiment and ten fluorescent and ten non-fluorescent colonies were picked from the protein G experiment. These colonies were expanded in a 24-well plate containing CD CHO medium and overgrowth titers were determined. As measured by IMMAGE instrument (Beckman Coulter), all fluorescent colonies produced antibody, while none of the non-fluorescent colonies produced antibody.

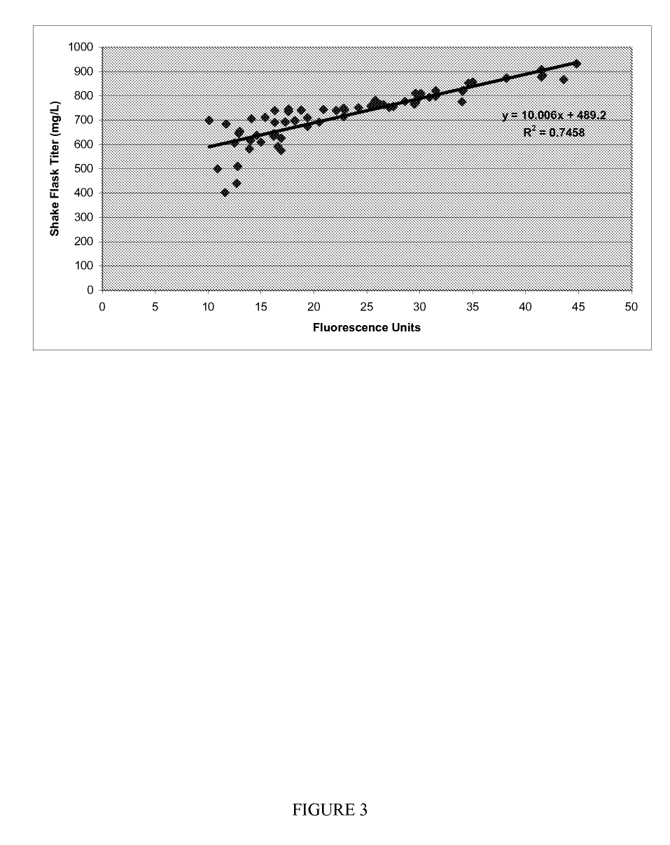

Another experiment was performed to determine correlation between fluorescence intensity and amount of expressed protein from selected clones. This experiment was setup using the abovementioned protocol with protein G (15 ug/mL) and a CHO cell line expressing a recombinant antibody (SM1.141). Mixed solution was plated in four 100 mm round culture dishes (10 mL volume). The plates were incubated undisturbed at 37° C. with 5% CO₂. After 13 days of incubation, a fluorescent microscope was used to visualize fluorescence. A total of 80 colonies were expanded in a 96 well plate containing 100 uL of CD CHO medium with 25 uM MSX and 1× GS supplement. Fluorescent pictures were taken of each colony with a digital camera connected to the microscope. Total fluorescence on or around a colony was quantitated using ImageJ software program from the National Institute of Health. Selected clones were expanded to batch shake-flask cultures in T25 flasks. Sixty-four clones that showed good growth in shake-flasks were seeded at 0.3×10⁶ cells/mL in 10 mL of CD CHO medium with 25 uM MSX and 1× GS supplement. Antibody titers were measured using the IMMAGE instrument after twelve days. Antibody titers and total fluorescence were plotted using Excel (Microsoft Corporation) to determine correlation (FIG. 3). Coefficient of Simple Determination (R²) is 0.75. If lower producing clones (<650 mg/L of Ab) are eliminated, Coefficient of Simple Determination approaches 0.88. These data indicate a strong correlation between total fluorescence and titer.

### Example 2

### Serum-Free, Animal Component-Free Fluorescent Protein G Screening Method for Cell Line Development

Fluorescent protein screening method was performed using serum-free, animal component-free conditions. The goal of these studies was to generate candidate cell lines expressing recombinant therapeutic proteins without exposure to any animal derived components. Recombinant protein expressing clones were isolated from serum-free, animal component-free methylcellulose plating using fluorescent protein G antibody secretion detection assay from both a primary transfection and sub-cloning of a high expression parental cell line.

### Use of Fluorescent Protein Screening to Isolate High Expression Sub-Clone Cell Lines Using, Serum-Free, Animal Component-Free Conditions

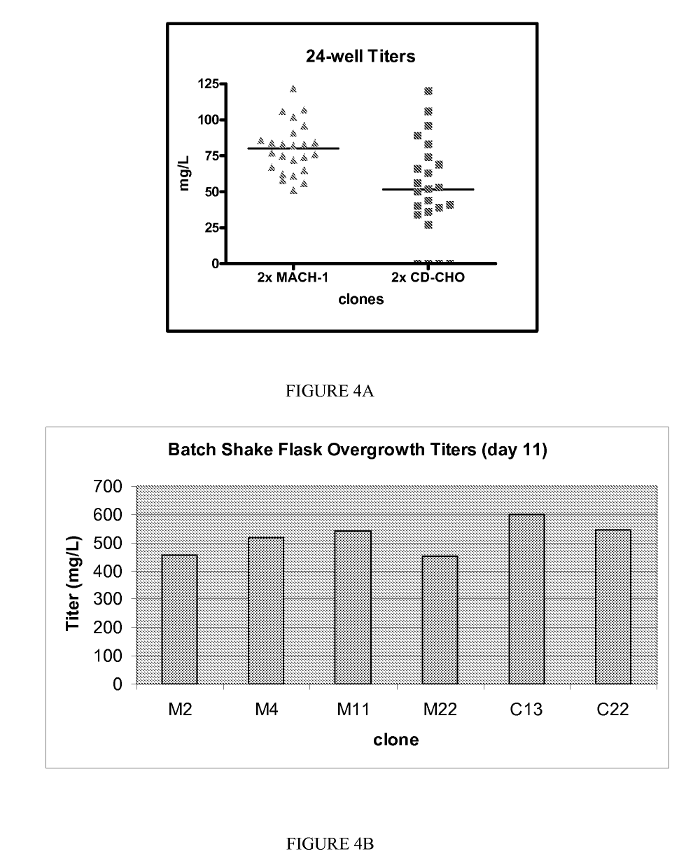

A parental CHOK1SV cell line expressing CNTO328 (KJ3.4D4) that was previously generated using the GS Gene Expression System (Lonza Biologics) was sub-cloned using the fluorescent protein G antibody secretion detection assay. Cells were plated at densities of 1000 or 2000 cells/mL in methylcellulose supplemented with either 2× CD-CHO or 2× MACH-1 (Table 6). Around 8-12 days post-plating, fluorescence intensity was visualized by microscopy. Approximately, 48 colonies from each condition with the highest fluorescence intensity picked and expanded to 24-well cultures for overgrowth titer determination (FIG. 4A). A total of six clones with the highest 24-well titers were expanded to shake flask cultures for overgrowth titer determination. Importantly, the titers for these top six sub-clone cell lines were determined to range from 450-600 mg/L (FIG. 4B). By comparison to previously reported data, the top sub-clone from parental cell line, KJ3.4D4, generated using the rabbit detection antibody immunoprecipitation method using 30% fetal bovine serum reached 570 mg/L.

TABLE 6
Reagents for serum-free, animal-component-free fluorescent protein G

| Component | Volume (mL) |
|---|---|
| CloneMatrix (Genetix) | 40 |
| XL Reagent (Genetix) | 2 |
| 50x GS supplement | 2 |
| Protein G (1 mg/mL reconstituted in PBS) | 15 |
| MSX (100 mM) | 0.025 |
| 2x CD-CHO or 2x MACH-1 | 41 |

methylcellulose screening

### Use of Fluorescent Protein G Screening to Isolate High Expression Parental Cell Lines Using, Serum-Free, Animal Component-Free Conditions

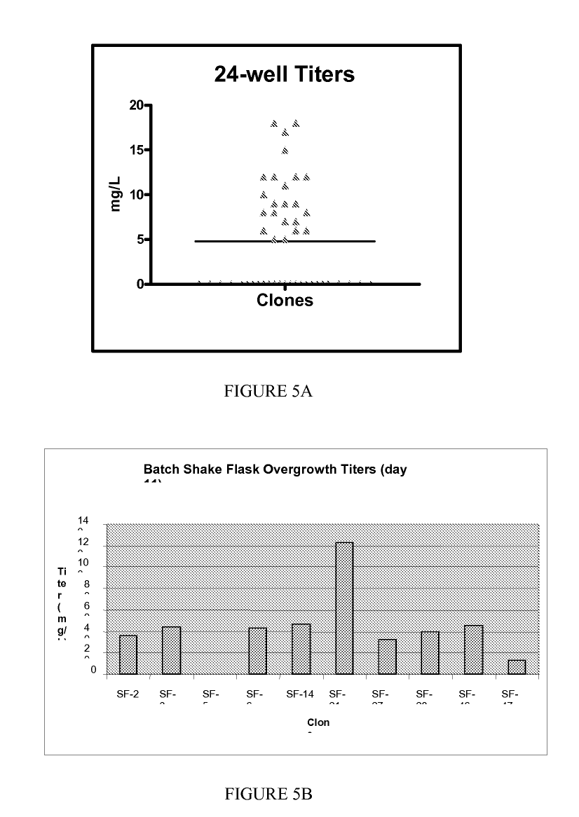

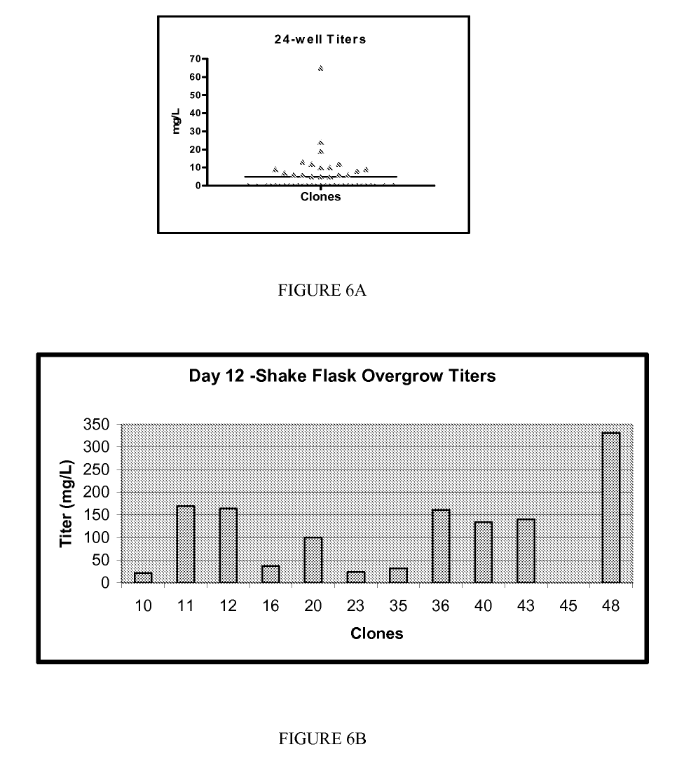

The host cell line CHOK1SV was electroporated with a GS CNTO1961 (chimeric anti-idiotypic antibody against test antibody) double gene plasmid. Transfected cells were recovered and selected in MACH-1 without fetal bovine serum. Cells were plated in methylcellulose at densities of either 20,000 or 40,000 cells/mL using the fluorescent protein G antibody secretion detection assay. Around 10-12 days post-plating, fluorescence intensity was visualized by microscopy. Densities of 20,000 cells/mL produced ˜2-5 clones/plate and those seeded at 40,000 cells/mL produced ˜10-15 clones/plate. Approximately 12-15 days post-plating a total of 48 colonies were picked into 96-well plate, irrespective of fluorescent intensity. All clones were expanded to 24-well cultures. 24-well overgrowth titers ranged from 0-18 mg/L (FIG. 5A). Based on 24-well titers, the top 10 highest expressing clones were selected for expansion to shake flasks. Shake flask overgrowth titers ranged from 0-120 mg/L (FIG. 5B). The same transfected and selected cells were plated in methylcellulose containing 30% fetal bovine serum and screened using the rabbit detection antibody immunoprecipitation method. The titers of 48 clones expanded to 24-well cultures ranged from 0-65 mg/L, including an outlier clone producing 65 mg/L (FIG. 6A). Batch shake flask overgrowth titers were determined for the top 10 cell lines ranged from 0-330 mg/L (FIG. 6B).

### SUMMARY

These studies demonstrate the utility of the fluorescent protein G screening assay for the identification and isolation of antibody producing parental clones using completely serum-free, animal component-free conditions. Moreover, sub-cloning in methylcellulose using the serum-free, animal component-free fluorescent protein G antibody secretion detection assay yielded sub-clones with titers comparable to those isolated using the rabbit detection antibody immunoprecipitation method.

### ADVANTAGES

Fluorescent protein A/G based secreted protein detection assay enables the detection and isolation of high-producer clones as the amount of fluorescence on or around a recombinant protein producing colony is directly proportional to the secreted protein. In comparison to methods utilizing rabbit antibodies, this method uses recombinant protein as the detection reagent and eliminates need to test for rabbit viruses on manufacturing cell lines. In addition, recombinant protein A/G is less expensive and lot-to-lot variations are lesser with recombinant protein reagents.

It will be clear that the invention can be practiced otherwise than as particularly described in the foregoing description and examples. Numerous modifications and variations of the present invention are possible in light of the above teachings and, therefore are within the scope of the appended claims.

What is claimed is:

1. A method for selecting high expression cell clones expressing at least one polypeptide of interest, comprising selecting at least one high expression cell clone from cells, cultured in a semi-solid culture medium comprising fluorescent protein A or G used for detection and expressing said polypeptide of interest, wherein the relative fluorescence of said bound fluorescent Protein A or G to said cells expressing said polypeptide of interest that interacts with said Protein A or G such that a relatively higher level of said fluorescence indicates higher relative expression of said polypeptide for each cell or group of cells.

2. A method according to claim 1, wherein said interaction is fluorescence of said Protein A or Protein G bound to said polypeptide.

3. A method according to claim 2, wherein said interaction is by a detectable label.

4. A method according to claim 3, wherein said detectable label is a fluorescent label.

5. A method according to claim 1, wherein the semi-solid culture medium comprises a gelatinization agent selected from cellulose or agar.

6. A method according to claim 5, wherein said cellulose is methylcellulose.

7. A method according to claim 1, wherein said cells are eukaryotic cells.

8. A method according to claim 7, wherein said eukaryotic cells are selected from mammalian cells, yeast cells or insect cells.

9. A method according to claim 7, wherein said mammalian cells are selected from COS-1, COS-7, HEK293, HK21, CHO, BSC-1, HepG2, 653, SP2/0, 293, NSO, DG44 CHO, CHO K1, HeLa, myeloma, or lymphoma cells, or any derivative, immortalized or transformed cells thereof.

10. A method according to claim 1, wherein said cells are prokaryotic cells.

11. A method according to claim 10, wherein said prokaryotic cells are bacterial cells or blue-green algae cells.

12. A method according to claim 1, wherein said at least one polypeptide of interest is a soluble polypeptide.

13. A method according to claim 1, wherein said at least one polypeptide of interest is an immunoglobulin or at least one portion thereof.

14. A method according to claim 1, wherein the cells are myeloma cells, said at least one polypeptide of interest is an immunoglobulin, the capture molecule is an antibody against the immunoglobulin, and the semi-solid culture medium is methylcellulose based.

15. A high expression cell clone, produced by a method according to claim 1.

16. A high expression cell clone according to claim 15, wherein the cells of said high expression cell clone are eukaryotic cells.

17. A high expression cell clone according to claim 16, wherein said eukaryotic cells are selected from mammalian cells, yeast cells or insect cells.

18. A high expression cell clone according to claim 17, wherein said mammalian cells are selected from COS-1, COS-7, HEK293, HK21, CHO, BSC-1, HepG2, 653, SP2/0, 293, NSO, DG44 CHO, CHO K1, HeLa, myeloma, or lymphoma cells, or any derivative, immortalized or transformed cells thereof.

19. A high expression cell clone according to claim 15, wherein said cells of said high expression cell clone are prokaryotic cells.

20. A high expression cell clone according to claim 19, wherein said prokaryotic cells are bacterial cells or blue-green algae cells.

21. A high expression cell clone according to claim 15, wherein said at least one polypeptide of interest is a soluble polypeptide.

22. A high expression cell clone according to claim 21, wherein said at least one polypeptide of interest is an immunoglobulin or at least one portion thereof.

23. A semi-solid culture medium to be used to identify a high expression cell clone expressing a polypeptide of interest, said medium comprising a cell growth culture medium and a gelatinization agent further comprising fluorescent protein A or G.

24. A semi-solid culture medium according to claim 23, wherein said gelatinization agent is selected from cellulose or agar.

25. A semi-solid culture medium according to claim 23, wherein said cellulose is methylcellulose.

26. A method according to claim 1, wherein said high expression cell clones are detectable by higher relative fluorescence of bound Protein A or G, relative to the cells having expression of said protein of interest that is lower than said high expression cell lines.

27. A method according to claim 1, wherein said at least one polypeptide of interest is selected from at least one of a growth factor, a cytokine, a blood protein, a neurotransmitter, pharmacologically active peptide, or any portion or derivative thereof.

28. A method according to claim 1, wherein said at least one polypeptide of interest is an antagonist of at least one selected from a growth factors, a cytokine, a blood protein, a neurotransmitter, and a pharmacologically active peptide.

29. A method according to claim 28, wherein said antagonist is selected from at least one of an antibody, an antibody fusion, an antibody fragment, or any portion thereof.

30. A method of claim 1, wherein said medium comprises animal free components.

31. A semi-solid culture medium of claim 23, wherein said medium comprises animal free components.
